(19) United States
(12) Patent Application Publication
(10) Pub. No.: US 2026/0207700 A1
(43) Pub. Date: Jul. 23, 2026

(54) MEDICAL PRODUCTS CONTAINING AN AQUEOUS FORMULATION OF A PEPTIDE

(71) Applicant: LYTIX BIOPHARMA AS, Oslo (NO)

(72) Inventors: Gry Stensrud, Nittedal (NO); Oystein Rekdal, Blommenholm (NO)

(21) Appl. No.: 19/140,523

(22) PCT Filed: Dec. 20, 2023
(86) PCT No.: PCT/EP2023/087127

§ 371 Date: Jun. 18, 2025

Related U.S. Application Data
(60) Provisional application No. 63/476,228, filed on Dec. 20, 2022.

(30) Foreign Application Priority Data
Jan. 30, 2023 (EP) 23153990.9
Mar. 14, 2023 (NO) 20230276

(51) Int. Cl. A61K 38/08; A61K 9/08; A61K 47/02; A61K 47/10; A61K 47/18; A61K 47/36

(52) U.S. Cl. CPC A61K 38/08; A61K 9/08; A61K 47/02; A61K 47/10; A61K 47/183; A61K 47/36

(57) ABSTRACT

The present invention relates to a medical product in the form of a sealed container containing an aqueous formulation of a peptide, or a pharmaceutically acceptable salt thereof, which: consists of 9 amino acids in a linear arrangement; of those 9 amino acids, 5 are cationic and 4 are lipophilic; and of these 4 lipophilic amino acids, 3 are tryptophan and 1 is non-genetically coded. Also provided are methods of making such products, wherein the aqueous formulation is introduced into the container and the filled container is mechanically sealed and methods of treatment utilising such products.

The present invention relates to medical products comprising peptides which are of use in cancer therapy. In particular, these products can be manufactured in dosage forms ready for use, i.e. ready for administration to the patient. These products therefore comprise the peptide in a stable formulation which can be transported and stored prior to use.

The prevalence of cancer in human and animal populations and its role in mortality means there is a continuing need for new therapies to combat tumours and tumour cells. Elimination of a tumour, a reduction in its size, the disruption of its supporting vasculature, or reducing the number of cancer cells circulating in the blood or lymph systems may be beneficial in a variety of ways; e.g. by reducing pain or discomfort, preventing metastasis, facilitating operative intervention or prolonging life.

Cancer therapies which are designed to combat tumours or cells metastasising from tumours typically rely on a cytotoxic activity. That activity might be a cytotoxic effect an active agent has itself or it might be an effect employed indirectly by the active agent, e.g. the upregulation of the host immune response against a tumour. To a greater or lesser extent these therapies are selective for the target (tumours or tumour cells) rather than normal healthy, or at least non-cancerous, cells and tissues. Those therapies that are poorly selective are associated with serious side effects as normal cells are exposed to the cytotoxic activity that the active agent relies upon to exert its therapeutic effect.

As described in patent publications WO2010/060497, WO2007/107748, WO2016/091487 and WO2016/091490, amphipathic peptides of 9 amino acids which contain cationic residues and tryptophan resides are highly effective anti-tumour agents. Not only do these peptides have a direct lytic action on tumour cells through disturbing and permeabilizing the cell membrane, they are also highly effective at attacking organelles such as mitochondria and lysosomes and can cause lysis thereof. This disruption of the organelle membrane results in the release of agents therefrom which have a potent immunostimulatory function, such agents are generally known as DAMPs (Damage-associated molecular pattern molecules) and include ATP, Cytochrome C, mitochondrial CpG DNA sequences, mitochondrial formyl peptides, cathepsins (from lysosomes) and HMGB1 (from the nucleus). As a consequence, these lytic peptides stimulate the immune system and provide an adaptive immune response which generates long term protection against tumour growth. The immune stimulation which occurs at the time the tumour antigens are released by the direct lytic effect on the tumour, results in enhanced antigen presentation and a type of in situ “vaccination” against the tumour.

One of these peptides is LTX-315, which is currently undergoing clinical trials for use as an anticancer agent. LTX-315 is currently provided in the form of its salts as a lyophilised (freeze-dried) powder product, with recommended storage under frozen or refrigerated conditions. LTX-315 is vulnerable to degradation by hydrolysis, which is generally addressed by the preparation of a product with non-aqueous solvents or by providing it as a dry, solid product, e.g. by freeze drying. Commercial suppliers have offered LTX-315 as a dry, solid product which needs to be stored below room temperature. For example, Aldrich Partner Products have offered LTX-315 as a powder product with recommended storage conditions in the dark, in an inert atmosphere, and in a freezer below-20° C. BOC Sciences have offered LTX-315 as a lyophilised powder product with recommended storage at −20° C. Amadis Chemical have also offered LTX-315 as a powder product with recommended storage in a tightly closed container, in a cool and dry place.

LTX-315 is generally intended to be administered by intratumoral injection and so the powder has to be dissolved, i.e. reconstituted, to be ready for administration, e.g. in saline to achieve an isotonic solution. Thus, the formulation for administration must be prepared in the clinical setting prior to use. Distribution as a lyophilised powder product which must be reconstituted prior to use is described in Koo, D. J et al. Biophysical Characterization of LTX-315 Anticancer Peptide Interactions with Model Membrane Platforms: Effect of Membrane Surface Charge. Int. J. Mol. Sci. 2022, 23, 10558, wherein the lyophilised LTX-315 peptide is said to be solubilised prior to use.

Additionally, the protocols for clinical trials involving LTX-315 carried out in 2021 and 2022 recommended that the lyophilized powder should be reconstituted with saline prior to use on-site. Preparation of injectable medicines (i.e. LTX-315 solutions for injection) in the clinical setting (on-site) carries significantly greater risk of medication error or microbiological contamination etc. than the preparation of injectable medicines outside of the clinical setting in a ready-to-use form. Moreover, the reconstitution process complicates the hospital logistics since it makes it more time consuming to prepare and administer the product. Therefore, if it was thought that LTX-315 could have been safely provided in a ready-to-use, aqueous formulation, this would have been chosen over the cumbersome and riskier on-site formulation preparation.

It was thus widely recognised in the art that it would not have been possible to provide ‘ready-to-use’ formulations of aqueous LTX-315.

There therefore exists a need for a convenient ready to use formulation of LTX-315 which could be manufactured, shipped and stored in a form which is suitable for direct administration.

The peptide sequence of LTX-315 using the one letter amino acid code is K-K-W-W-K-K-W-Dip-K-NH₂, in which Dip=diphenylalanine. LTX-315 comprises several tryptophan (W) residues which, within a peptide, are known to pose a significant hurdle for chemical stability of the peptide in aqueous solution. The tryptophan side chain may undergo a plethora of chemical reactions in aqueous solution, caused by exposure to light, reactive oxygen and to some extent changes in pH, increased temperature, or exposure to metal cations, which may be extractables/leachables from primary packaging materials such as rubber stoppers and plungers [Bellmaine S, Schnellbaecher A, Zimmer A. Reactivity and degradation products of tryptophan in solution and proteins. Free Radic Biol Med. 2020; 160:696-718. doi: 10.1016/j.freeradbiomed.2020.09.002, and Schöneich C. Novel chemical degradation pathways of proteins mediated by tryptophan oxidation: tryptophan side chain fragmentation. J Pharm Pharmacol. 2018; 70(5):655-665. doi:10.1111/jphp.12688].

Short peptides lack the ability to form stabilising secondary structures in solution. Thus, such poor physical stability and known chemical degradation pathways of similar short peptides have led to an understanding in the art that a relatively short peptide such as LTX-315 would be unstable in aqueous solution over the time period from its manufacture to the administration to a patient. A notable chemical degradation pathway involving a tryptophan sidechain, which may be expected to be relevant for LTX-315, is observed in the synthetic peptide octreotide, an octapeptide, which is a modified analogue of somatostatin (National Center for Biotechnology Information (2022). PubChem Compound Summary for CID 448601, Octreotide. Retrieved Jun. 14, 2022 from https://pubchem.ncbi.nlm.nih.gov/compound/Octreotide). In octreotide, the indole moiety of a D-tryptophan is transferred to the adjacent lysine (K) sidechain through a complex reaction pathway initiated by exposure to UV light. The former D-tryptophan amino acid is then transformed into a hydroxyglycine and subsequently the peptide chain is cleaved into two fragments. LTX-315 has one motif with W-K in the same order as in octreotide and two additional K-W motifs.

The octreotide drug product is formulated only up to 1.0 mg/ml and at around pH 4.0 (the isoelectric point is 8.3). This pH would not be preferred for intratumoral injection (a pH of 5-7 is generally acceptable for intratumoral injection). Two liquid formulation products of octreotide exist, either for single-use or in a phenol-containing, preserved formulation for multiple-use. Though the product may be stored between 20-30° C. (protected from light) this is only for up to 14 days, which indicates that the overall stability of this short peptide is limited in an aqueous formulation. Moreover, octreotide is a cyclised peptide, which generally improves both physical and chemical stability in comparison with a linear peptide.

Another potential chemical degradation pathway for LTX-315 is the formation of intermolecular ditryptophan crosslinks or bridges through oxidation. Formation of covalent LTX-315 dimers through a ditryptophan crosslink would be regarded as an undesirable degradation product. The potential of ditryptophan formation is also highlighted through comparison with indolicidin. In this 13-residue peptide a ditryptophan covalent cross-link between tryptophan residues 6 and 9, i.e. the same distance as between tryptophan residues 5 and 8 in LTX-315, was characterized. Although this crosslink was formed under peptide synthesis conditions in TFA, it shows that such a geometry/distance in the sequence allows formation of intramolecular Trp-Trp crosslinks under the right conditions [Osapay K, Tran D, Ladokhin A S, White S H, Henschen A H, Selsted M E. Formation and characterization of a single Trp-Trp cross-link in indolicidin that confers protease stability without altering antimicrobial activity. J Biol Chem. 2000; 275(16):12017-12022. doi:10.1074/jbc.275.16.12017].

Comparison can also be made with degarelix, a decapeptide, which is marketed as a lyophilized powder, indicating stability labilities if formulated in an aqueous solution. A peptide of 9 or 10 amino acids is generally too short to form secondary structures which would enhance stability.

Thus, it is clear that with multiple tryptophan residues, and being only 9 amino acids in length, LTX-315 and similar peptides would be considered by the person skilled in the art to be vulnerable to degradation, particularly in an aqueous formulation. This has led to a lyophilised powder product being the standard preparation of LTX-315 with a recommendation to store the powder product in a freezer or refrigerator in order to prevent degradation of the peptide.

However, the inventors have surprisingly found that peptides such as LTX-315 and pharmaceutically acceptable salts thereof are in fact stable in aqueous solution, this means a peptide containing product can be manufactured in dosage forms ready for use, i.e. ready for direct administration to the patient. These products comprise the peptide in a stable form which can be transported and stored prior to use.

The present invention therefore provides a medical product in the form of a sealed container containing an aqueous formulation of a peptide or a pharmaceutically acceptable salt thereof, which:
a) consists of 9 amino acids in a linear arrangement;
b) of those 9 amino acids, 5 are cationic and 4 are lipophilic; and
c) of these 4 lipophilic amino acids (i.e. amino acids with a lipophilic R group), 3 are tryptophan and 1 is non-genetically coded; and optionally
d) the lipophilic and cationic residues are arranged such that there are no more than two of either type of residue adjacent to one another; and further optionally
e) the molecule comprises two pairs of adjacent cationic amino acids and one or two pairs of adjacent lipophilic residues.

Such formulations are suitable for direct administration to a patient, e.g. intratumourally, e.g. by injection or by perfusion/infusion. This offers improved convenience to the clinician who does not need to make up the formulation for administration to the patient, e.g. by dissolving a powdered form of the peptide in a suitable solvent.

### The Peptide

The cationic amino acids, which may be the same or different, are preferably lysine or arginine but may be histidine or any non-genetically coded or modified amino acid carrying a positive charge at pH 7.0. Lysine is especially preferred.

Suitable non-genetically coded cationic amino acids and modified cationic amino acids include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

The non-genetically coded lipophilic amino acid has an R group with at least 7, preferably at least 8 or 9, more preferably at least 10 non-hydrogen atoms. An amino acid with a lipophilic R group is referred to herein as a lipophilic amino acid or lipophilic residue. Typically the lipophilic R group has at least one, preferably two cyclic groups, which may be fused or connected.

The lipophilic R group may contain hetero atoms such as O, N or S but typically there is no more than one heteroatom, preferably it is nitrogen. This R group will preferably have no more than 2 polar groups, more preferably none or one, most preferably none.

As disclosed herein, a D amino acid, while not strictly genetically coded, is not considered to be a “non-genetically coded amino acid”, which should be structurally, not just stereospecifically, different from the 20 genetically coded L amino acids. The molecules disclosed herein may have some or all of the amino acids present in the D form.

The non-genetically coded lipophilic amino acid preferably has an R group which contains no more than 30 non-hydrogen atoms, more preferably no more than 25 non-hydrogen atoms.

Preferred non-genetically coded amino acids include: 2-amino-3-(biphenyl-4-yl)propanoic acid (biphenylalanine), 2-amino-3,3-diphenylpropanoic acid (diphenylalanine), 2-amino-3-(anthracen-9-yl)propanoic acid, 2-amino-3-(naphthalen-2-yl)propanoic acid, 2-amino-3-(naphthalen-1-yl)propanoic acid, 2-amino-3-[1,1′:4′,1″-terphenyl-4-yl]-propionic acid, 2-amino-3-(2,5,7-tri-tert-butyl-1H-indol-3-yl)propanoic acid, 2-amino-3-[1,1′:3′,1″-terphenyl-4-yl]-propionic acid, 2-amino-3-[1,1′:2′,1″-terphenyl-4-yl]-propionic acid, 2-amino-3-(4-naphthalen-2-yl-phenyl)-propionic acid, 2-amino-3-(4′-butylbiphenyl-4-yl)propanoic acid, 2-amino-3-[1,1′:3′,1″-terphenyl-5′-yl]-propionic acid and 2-amino-3-(4-(2,2-diphenylethyl)phenyl)propanoic acid. Diphenylalanine and biphenylalanine are especially preferred.

In a preferred embodiment the peptide has one of formulae (I) to (V) listed below, in which C represents a cationic amino acid as defined above and L represents a lipophilic amino acid as defined above (i.e. tryptophan (Trp/W) or a non-genetically coded lipophilic amino acid). The amino acids are covalently linked by peptide bonds. The free amino or carboxy terminals of these molecules may be modified, the carboxy terminus is preferably modified to remove the negative charge, most preferably the carboxy terminus is amidated, this amide group may be substituted.

(I)
(SEQ ID NO: 1)
CCLLCCLLC
(II)
(SEQ ID NO: 2)
LCCLLCCLC
(III)
(SEQ ID NO: 3)
CLLCCLLCC
(IV)
(SEQ ID NO: 4)
CCLLCLLCC
(V)
(SEQ ID NO: 5)
CLCCLLCCL

β and γ amino acids as well as a amino acids are included within the term ‘amino acids’, as are N-substituted glycines.

As discussed above, the peptides incorporate one non-genetically coded lipophilic amino acid. When this residue is denoted L′, preferred peptides are represented by the following formulae:

(I′)
(SEQ ID NO: 6)
CCL′LCCLLC
(I″)
(SEQ ID NO: 7)
CCLLCCLL′C
(I″)
(SEQ ID NO: 8)
CCLL′CCLLC
(II′)
(SEQ ID NO: 9)
LCCLL′CCLC

Particularly preferred are peptides of formula (I) and (II), and of these, peptides of formula (I″) are especially preferred.

The following peptides are most preferred:

| | SEQ | |
|---|---|---|
| | ID | |
| Name | NO | Sequence |
| LTX-301 | 10 | Dip-K-K-W-W-K-K-W-K-NH₂ |
| LTX-302 | 11 | W-K-K-W-Dip-K-K-W-K-NH₂ |
| LTX-303 | 12 | W-K-K-W-W-K-K-Dip-K-NH₂ |
| LTX-304 | 13 | Bip-K-K-W-W-K-K-W-K-NH₂ |
| LTX-305 | 14 | W-K-K-Bip-W-K-K-W-K-NH₂ |
| LTX-306 | 15 | w-k-k-w-dip-k-k-w-k-NH₂ |
| LTX-307 | 16 | K-K-W-Dip-K-K-W-W-K-NH₂ |
| LTX-308 | 17 | k-k-W-Dip-k-k-W-W-k-NH₂ |
| LTX-309 | 18 | K-K-W-Dip-K-K-W-Dip-K-NH₂ |
| LTX-310 | 19 | K-K-W-Bip-K-K-W-W-K-NH₂ |
| LTX-312 | 20 | K-Bip-K-K-W-W-K-K-W-NH₂ |
| LTX-313 | 21 | K-K-Bip-W-K-K-W-W-K-NH₂ |
| LTX-314 | 22 | K-K-W-W-K-K-Dip-W-K-NH₂ |
| LTX-315 | 23 | K-K-W-W-K-K-W-Dip-K-NH₂ |
| LTX-316 | 24 | K-W-Dip-K-K-W-W-K-K-NH₂ |
| LTX-317 | 25 | K-K-W-W-K-W-Dip-K-K-NH₂ |
| LTX-318 | 26 | Orn-Orn-W-Dip-Orn-Orn-W-W-Orn-NH₂ |
| LTX-319 | 27 | Dap-Dap-W-Dip-Dap-Dap-W-W-Dap-NH₂ |
| LTX-320 | 28 | R-R-W-Dip-R-R-W-W-R-NH₂ |
| LTX-321 | 29 | K-W-W-K-K-Dip-W-K-K-NH₂ |
| LTX-323 | 30 | K-Dip-K-K-W-W-K-K-W-NH₂ |
| LTX-324 | 31 | K-K-Dip-W-K-K-W-W-K-NH₂ |
| LTX-325 | 32 | k-w-w-k-k-dip-w-k-k-NH₂ |
| LTX-326 | 33 | R-R-Bip-W-R-R-W-W-R-NH₂ |
| LTX-327 | 34 | R-R-Dip-W-R-R-W-W-R-NH₂ |
| LTX-329 | 35 | k-k-bip-w-k-k-w-w-k-NH₂ |
| LTX-331 | 36 | k-k-Bip-w-k-k-w-w-k-NH₂ |
| LTX-332 | 37 | K-K-bip-W-K-K-W-W-K-NH₂ |
| LTX-333 | 38 | Dab-Dab-W-Dip-Dab-Dab-W-W-Dab-NH₂ |
| LTX-334 | 39 | K-K-W-1-Nal-K-K-W-W-K-NH₂ |
| LTX-335 | 40 | K-K-W-2-Nal-K-K-W-W-K-NH₂ |
| LTX-336 | 41 | K-K-W-Ath-K-K-W-W-K-NH₂ |
| LTX-338 | 42 | K-K-W-Phe(4-4′Bip)-K-K-W-W-K-NH₂ |

In which:
the standard single letter code is used for the genetically coded amino acids
lower case denotes D amino acids
Dip is diphenylalanine
Bip is biphenylalanine
Orn is ornithine
Dap is 2,3-diaminopropionic acid
Dab is 2,4-diaminobutyric acid
1-NaI is 1-naphthylalanine
2-NaI is 2-naphthylalanine
Ath is 2-amino-3-(anthracen-9-yl)propanoic acid
Phe(4,4′Bip) is 2-amino-3-[1,1′:4′,1″-terphenyl-4-yl]propionic acid

Preferred peptides are LTX-302, LTX-313, LTX-315, LTX-320 and LTX-329. Said preferred peptides are preferably in the form of a pharmaceutically acceptable salt, preferably an acetate salt.

For use in the invention, all of the peptides described herein may be in the form of a pharmaceutically acceptable salt. The peptides preferably have a modified, particularly an amidated, C-terminus. Suitable pharmaceutically acceptable salts are well known in the art and include salts of inorganic or organic acids, and include hydrochloride, trifluoroacetate and acetate salts. Acetate salts are most preferred.

Especially preferred is the peptide known as LTX-315, in particular in the form of a pharmaceutically acceptable salt, preferably in the form of its acetate salt.

Thus in a preferred embodiment the present invention provides a medical product in the form of a sealed container containing an aqueous formulation of LTX-315 having the amino acid sequence of SEQ ID NO: 23, or a pharmaceutically acceptable salt thereof, preferably the acetate salt thereof.

### Synthesis of the Peptide

The peptides described herein such as LTX-315 and its pharmaceutically acceptable salts may be synthesised in any convenient way by methods of peptide synthesis well known in the art, preferably by carrying out the synthesis on a solid phase support. Methods of synthesis are for example described in WO2010/060497, WO2016/091487 and WO2016/091490.

### Aqueous Formulation

As discussed above, the medical product of the present invention is in the form of a sealed container containing an aqueous formulation of the peptide as defined herein. In the following discussion, the term “peptide” denotes the peptide and its pharmaceutically acceptable salts.

The sealed container may contain only the aqueous formulation of the peptide as defined herein. In other embodiments, the sealed container is not completely filled with the aqueous formulation of the peptide as defined herein, and the non-filled volume (the headspace) contains air or an inert gas such as nitrogen or argon.

In some embodiments, the aqueous formulation comprises the peptide at a concentration of from 1 to 30 mg/ml, preferably 4 to 20 mg/ml, or more preferably 6 to 16 mg/ml. The afore-mentioned amounts of peptide refer to the net peptide amount (see Example section for a description of net peptide amount).

An aqueous formulation is one in which the solvent for dissolving the peptide is water, i.e. a solution of the peptide in water.

Since the peptide is readily soluble in water, and an aqueous formulation of it does not need to comprise an organic co-solvent for dissolving the peptide, the formulation preferably does not comprise an organic co-solvent for dissolving the peptide. Indeed, there may be no organic components at all in the aqueous formulation (other than the peptide), although, as discussed below, organic components may be included, e.g. as preservative agents, viscosity enhancers, or stabilizers.

The aqueous formulation may consist only of water and the peptide, i.e. the aqueous formulation is a solution of the peptide in water, such as water for injection (WFI). Alternatively, the aqueous formulation may comprise or consist of water, the peptide and a salt (e.g. sodium chloride), resulting in a saline solution of the peptide. In some other embodiments, the aqueous formulation comprises or consists of the peptide and an aqueous buffer, i.e. is a solution of the peptide in an aqueous buffer. In some embodiments, the aqueous buffer is a mixture of several different aqueous buffers. The aqueous formulation preferably comprises a solution of the peptide in an aqueous buffer and optionally one or more further excipients as discussed below.

In some embodiments, the aqueous formulation does not include preservative agents and/or stabilizers. In some embodiments, the aqueous formulation does not include hydroxybenzoates and/or EDTA.

Optionally, the aqueous formulation may comprise one or more further pharmaceutically acceptable ingredients, i.e. excipients, such as those used in medical products comprising aqueous formulations of active ingredients, such excipients are well known in the art. Suitable excipients are those used in parenteral formulations, for example formulations administered by intratumoral injection, a preferred method of administration in the context of the present invention.

For example, one or more preservative agents may be included in the aqueous formulation, particularly if the sealed container contains multiple doses. The table below lists examples of preferred preservative agents. Also listed are suitable concentration ranges for these agents if present in the aqueous formulation:

| Preservative agent | Concentration (w/w) |
|---|---|
| Benzalkonium chloride | 0.004% to 0.02% |
| Benzyl alcohol | 0.75 to 5% |
| Chlorobutanol | 0.25 to 0.5% |
| Metacresol (m-cresol) | 0.1 to 0.35% |
| Methylparaben | 0.01% to 0.2% |
| Phenol | 0.15 to 0.5% |
| Potassium sorbate | 0.05% to 0.2% |
| Propylparaben | 0.005 to 0.035 |
| Thimerosal | 0.001% to 0.012% |

Benzyl alcohol is a particularly preferred preservative agent, preferably this is present at 0.75% to 2% w/w, preferably 1% w/w. Another preferred preservative agent is phenol, which is preferably present at 0.15% to 0.5%.

One or more chelating agents may be included to chelate metal ions. The table below lists examples of preferred chelating agents. Also listed are suitable concentration ranges for these agents if present in the aqueous formulation:

| Chelating Agents | Concentration (w/w) |
|---|---|
| Calcium disodium EDTA | 0.01 to 0.1% |
| Calcium versetamide sodium | 2 to 3.5% |
| Calteridol | 0.01 to 0.03% |
| Disodium EDTA | 0.01 to 0.11% |
| DTPA | 0.04 to 1.2% |
| Sodium EDTA | 0.1 to 0.3% |

EDTA = ethylenediaminetetraacetic acid,
DTPA = diethylenetriamine pentaacetate

Preferably the chelating agent is disodium EDTA and/or sodium EDTA. Preferably the chelating agent, such as disodium or sodium EDTA, is present at about 0.1% w/w.

One or more antioxidants may be included. The table below lists examples of preferred antioxidants. Also listed are suitable concentration ranges for the antioxidants if present in the aqueous formulation:

| Antioxidants | Concentration (w/w) |
|---|---|
| Ascorbic Acid | 0.02 to 0.15% |
| Ascorbyl palmitate | 0.01 to 0.03% |
| Citric acid | 0.10 to 2.0% |
| Erythorbic acid | 0.02 to 0.1% |
| Methionine | 0.01 to 0.15% |
| Monothioglycerol | 0.1 to 1% |
| Potassium metabisulfite | 0.05% to 0.10% |
| Sodium ascorbate | 0.1 to 4.8% |
| Sodium bisulfite | 0.1 to 0.15% |
| Sodium formaldehyde sulfoxylate | 0.1 to 0.15% |
| Sodium thiosulfate | 0.1 to 0.2% |
| Thiourea | 0.002 to 0.008% |

A preferred antioxidant is thiourea.

As mentioned above, in some embodiments, the aqueous formulation is a solution of the peptide in an aqueous buffer or in a mixture of aqueous buffers. Buffers may be used to maintain the pH of the aqueous formulation within the desired range. Preferably, the aqueous formulation has a pH of from 5.0 to 7.4, more preferably from 5.0 to 7.0, and even more preferably from 5.0 to 6.0 or 6.5. The following aqueous buffers, amongst others, are therefore preferred: acetic acid, acetate, aspartic acid, sodium benzoate, benzoic acid, carbonic acid, citric acid, glucono-delta-lactone, glycine, glycine HCl, histidine, histidine HCl, hydrobromic acid, phosphate (such as PBS), sodium succinate, disodium succinate, succinic acid, sulfuric acid, tartaric acid and/or sodium tartrate. Preferably the aqueous buffer is an acetic acid/acetate buffer, citric acid buffer and/or a phosphate buffer.

One or more tonicity modifiers may be included, such as dextrose, glycerine, lactose, mannitol, potassium chloride, sodium chloride and/or sorbitol, to obtain an isotonic aqueous formulation which reduces pain experienced by a patient receiving the aqueous formulation via parenteral administration. Preferably the tonicity modifier is sodium chloride.

In some embodiments, one or more stabilizers which inhibit aggregation of the peptide are included in the aqueous formulation. Such stabilizers include anti-stacking agents. Examples of suitable stabilizers are: dimethyl sulfoxide, dimethylacetamide, ethanol, glycerol, mannitol, N-methyl-2-pyrrolidone, polyethylene glycol (PEG) 200, PEG 300, PEG 350, PEG 400, PEG 600, propylene glycol and sorbitol. PEG stabilizers, e.g. PEG 200, are particularly preferred. Such stabilizers, e.g. PEG stabilizers, may be present at 1 to 8% w/w, preferably 2 to 7% w/w, e.g. 3 to 6% w/w.

The aqueous formulation may further comprise one or more viscosity enhancers, such as acacia gum, agar, cellulose derivatives, such as methylcellulose, hydroxypropyl cellulose, hydroxypropyl methylcellulose (HPMC), hydroxyethyl cellulose, carboxymethylcellulose calcium, carboxymethylcellulose sodium, chitosan, gelatine, hyaluronic acid, maltodextrin, pectin, poloxamer 407, PEG, polyethylene oxide, povidone (PVP), starches like corn starch and tapioca starch, tragacanth and/or β-cyclodextrins.

Enhanced viscosity (compared to that of water) of the aqueous formulation can effectively slow the dispersion of the peptide from the site of administration. If the formulation is administered intratumorally, enhanced viscosity can extend the time the formulation stays within the tumour and does not leak out, slowing dispersion of the peptide by the blood and prolonging the time the peptide is in direct contact with the tumour cells. In this way, the efficacy of the peptide may be increased and/or the toxicity reduced.

Thus in some embodiments, the aqueous formulation comprises one or more viscosity enhancers and has a viscosity of from 1.5, 2, 2.5 or 3 cP to 300 cP, e.g. from 2.5 to 250 cP, such as 5 to 100 cP or 10 to 200 cP and 20 to 240 cP, preferably of from 40 to 180 cP, e.g. from 45 to 150 cP or from 30 to 100 cP and more preferably 1.5 to 10 cP, such as 2, 3, 4, 5, 6, 7, 8, 9 or 10 cP or of from 10 to 15 cP or 15 to 20 cP or 20 to 35 cP or 35 to 50 cP or 50 to 100 cP.

In some embodiments, wherein the medical product is a pre-filled syringe or pre-filled syringe cartridge, and wherein the viscosity of the aqueous formulation of the medical product is above 250 cP, above 200 cP, above 150 or above 100 cP, specialist needles may be used.

Preferably the one or more viscosity enhancers are selected from the group consisting of chitosan or a chitosan derivative or a pharmaceutically acceptable salt thereof, such as a hydrochloride salt, hydroxyethyl cellulose, hydroxypropyl cellulose, HPMC, poloxamer 407, PEG, and PVP. HPMC and chitosan are particularly preferred viscosity enhancers. The chitosan may be very low molecular weight chitosan (typically <50 kDa), low molecular weight chitosan (typically 50 to <100 kDa), medium molecular weight chitosan (typically 100-1000 kDa) or high molecular weight chitosan (typically >1000 kDa). In some embodiments, the chitosan has a degree of deacetylation (DDA) of about 50 to 99%, such as 70 to 99%, such as 80%, preferably of about 70% and above.

Chitosan contains active functional groups that are liable to chemical reactions and the modification of chitosan by e.g. acylation, carboxylation, alkylation, and quaternization leads to chitosan derivatives with properties different from chitosan, such as improved solubility or bioactivity, but retaining the unique original pharmacological properties of chitosan (see Wang et al. Int. J. Mol. Sci 21(2), 2020, 487, doi: 10.3390/ijms21020487).

In some embodiments, the viscosity enhancer is a chitosan derivative, such as O- and N-acylated chitosan, O- and N-alkylated chitosan, chitosan modified by introduction of a hydrophilic group, such as a carboxylic acid group, quaternary ammonium group, sulfonic acid group, phosphoric acid group, amino group, ether bonds composed of an oxygen group, hydroxyl group, carboxylate group, and block polyether group.

In some embodiments, the viscosity enhancer is a glycated chitosan, obtained by reaction of free amino groups of chitosan and carbonyl groups of reducing monosaccharides and/or oligosaccharides. Examples of such monosaccharides include naturally occurring D-trioses, D-tetroses, D-pentoses, D-hexoses, D-heptoses, and the like, such as D-glucose, D-galactose, D-fructose, D-mannose, D-allose, D-altrose, D-idose, D-talose, D-fucose, D-arabinose, D-gulose, D-hammelose, D-lyxose, D-ribose, D-rhamnose, D-threose, D-xylose, D-psicose, D-sorbose, D-tagatose, D-glyceraldehyde, dihydroxyacetone, D-erythrose, D-threose, D-erythrulose, D-mannoheptulose, D-sedoheptulose and the like. Examples of such oligosaccharides include fructo-oligosaccharides (FOS), galacto-oligosaccharides (GOS), mannan-oligosaccharides (MOS) and the like. An example of a glycated chitosan is galactochitosan. Glycated chitosan and its synthesis is described for instance in U.S. Pat. No. 5,747,475 A and WO 2002/040055 A2 and WO 2013/109732 A2, the content of which is incorporated herein by reference.

In some embodiments, the viscosity enhancer is glycated chitosan which possesses from about 0.1% to about 90% percent glycation of its otherwise free amino groups, such as from about 0.1% to about 30% or such as from about 2 to 15%, such as about 12.5%. In some embodiments, the glycated chitosan has a molecular weight from about 50 kD to about 2000 kD, such as from about 50 kD to about 1500 kD, such as about 250 kD or 300 kD. In some embodiments, the glycated chitosan has a DDA of about 50 to 99%, such as 70 to 99%, such as 80%. In some embodiments, the glycated chitosan has a DDA of about 50 to 99%, such as 70 to 99%, such as 80%, possesses from about 0.1% to about 90% percent glycation of its otherwise free amino groups, such as from about 0.1% to about 30% or such as from about 2 to 15%, such as about 12.5% and has a molecular weight from about 50 kD to about 2000 kD, such as from about 50 kD to about 1500 kD, such as about 250 kD or about 300 kD.

Viscosity enhancers are preferably present at 0.1 to 5% w/w, e.g. 0.15 to 2.5% w/w or 0.2 to 2% w/w or 0.25 to 1.5% w/w or 0.3 to 1% w/w or 0.35 to 0.9% w/w. In some embodiments, the viscosity enhancer is chitosan or a chitosan derivative which is present at 0.1 to 5% w/w, such as 0.1 to 2.5% w/w such as 0.5%, 1%, 1.5% or 2% w/w.

For example, using commercially available low molecular weight HPMC products as viscosity enhancers, at a concentration of 0.5% w/w in the aqueous formulation, viscosities of about 35 to 50 cP have been demonstrated, while using commercially available high molecular weight HMPC products at a concentration of 0.5% w/w, viscosities of about 145 to 200 cP have been demonstrated.

Alternatively, using commercially available low molecular weight chitosan products as viscosity enhancers, at concentrations of 1 to 1.5% w/w in the aqueous formulation, viscosities in the range of about 35 to 110 cP have been demonstrated, while using commercially available medium molecular weight chitosan product at concentrations of 1 to 1.5% w/w, viscosities in the range of about 50 to 175 cP have been demonstrated.

The aqueous formulation may comprise a viscosity enhancer in admixture with one or more further excipients, e.g. a preservative agent or a chelating agent.

The one or more optional further excipients (i.e. other than the peptide and water) discussed above, are preferably present in the aqueous formulation in the medical product of the invention at an individual component concentration of no more than 10% w/w, no more than 9% w/w, no more than 8% w/w, no more than 7% w/w, or no more than 6% w/w, preferably no more than 5% w/w, no more than 4% w/w, no more than 3% w/w, or no more than 2% w/w or no more than 1% w/w.

Preferably the total amount of excipients present in the aqueous formulation in the medical product of the invention is no more than 20% w/w, no more than 10% w/w, preferably no more than 5% w/w, more preferably, no more than 4% w/w, e.g. no more than 3% w/w or no more than 2% w/w.

In addition, if any organic excipients are present, the total amount of all such organic excipients in the aqueous formulation in the medical product of the invention is less than 10% w/w, less than 9% w/w, less than 8% w/w, less than 7% w/w, or less than 6% w/w, preferably less than 5% w/w, less than 4% w/w, less than 3% w/w, or less than 2% w/w or less than 1% w/w.

The w/w values are calculated for the aqueous formulation excluding the peptide itself, i.e. for the excipients in the formulation.

### Medical Product

The medical product of the present invention is a pharmaceutical grade product. The medical product contains the peptide in a ready to use form which can be utilised directly by the end user without the need to add/mix with any other ingredients prior to administering it to a patient in need thereof. In some embodiments, the aqueous formulation is a sterile formulation.

The medical product is in the form of a sealed container, in particular the product of the invention incorporates a sterile seal, thus the aqueous formulation within the sealed container is kept under sterile conditions within the container. The seal is watertight and may be substantially or completely airtight. In the products of the invention, the integrity of the seal may be maintained, is capable of being maintained, for at least 3, 6, 9, 12, 18, 24 or 48 months.

Preferably the seal is a manufactured seal; this is in contrast to a bung or lid which may be applied to a container of the formulation by an individual after having generated the formulation from a solid form of the peptide. Thus the seal may be considered to be formed in an industrial/mechanical process.

The aqueous formulation comprising the peptide is usually administered by injection. Sites of injection include intratumoural, dermal, intralesional (e.g. in the case of cancerous moles), subcutaneous, intravenous, intracavity (e.g. intraperitoneal). The formulation may be administered in other ways, e.g. topically, by perfusion or infusion or transdermally. Intratumoural and intralesional modes of administration are preferred.

The medical product of the invention thus preferably is a sealed container containing a ready-to-inject aqueous formulation of the peptide, preferably a ready-to-inject aqueous solution of the peptide. The medical product may, in preferred embodiments, be a pre-filled syringe or pre-filled syringe cartridge. A pre-filled syringe medical product of the invention may comprise a needle or, in the case of a cartridge, will need to have a needle fixed thereto. Such devices are well known in the art and are increasingly popular as they allow for precise dosing, reduce waste and are easy for the user (e.g. a clinician, nurse or patient themselves) to prepare for injection (Prefilled syringes: An innovation in parenteral packaging, Makwana et al, Int J Pharm Investig. 20122 1(4) pp 200-206). Typically, in contrast to a mixed-for-use formulation in a syringe, a pre-filled syringe (or cartridge) can remain sterile for an indefinite period of time, provided that its integrity is not compromised.

A pre-filled syringe medical product will usually incorporate a cap on the needle or on the tip to maintain the seal on the barrel containing the aqueous formulation; at the other end it will typically have a plunger attached to a stopper. A cartridge may be sealed with a Luer lock or slip tip type closure. The cartridge may or may not comprise a plunger, thus it may comprise a dosed and sealed barrel which is adapted to fit into a syringe housing which has both a plunger and a needle.

The barrel which contains the aqueous formulation in a pre-filled syringe or syringe cartridge may be made of glass or plastic. In some embodiments the glass may be a pharmaceutical type 1 glass. In other embodiments the plastic may be cyclic olefin polymer, cyclic olefin copolymer, polypropylene or polycarbonate.

In a preferred embodiment, the present invention provides a pre-filled and sealed syringe or syringe cartridge containing an aqueous formulation of LTX-315, having the amino acid sequence of SEQ ID NO. 23, preferably the form of a pharmaceutically acceptable salt, such as the acetate salt thereof.

Other suitable sealed containers include ampoules and vials. Both of these sealed containers are convenient ways to transport and store aqueous formulations of the peptides defined herein which are ready to use. Both are also highly suitable for facilitating delivery by injection. An ampoule is typically for one time use as the integral cap (e.g. of glass) is broken off to allow access (e.g. by a needle) to the aqueous formulation therein. Vials may contain larger volumes of the aqueous formulation and incorporate a fixed cover or stopper (bung) which can be penetrated by a needle (e.g. made of rubber or an equivalent synthetic material). This stopper may conveniently be surrounded by a crimp cap (e.g. made of metal such as aluminium) and optionally covered with a plastic cap. The use of a penetrable stopper means the vial can contain multiple dosages with the vial still being sealed as the rubber effectively closes off the “pore” generated by the needle.

The sealed container may have a useable volume from 0.1 to 20 ml, such as from 0.2 to 10 ml or from 0.3 to 8 ml, preferably from 0.5 to 5 ml. Therefore, a sealed container with a useable volume of 2 ml, containing an aqueous formulation comprising 2 ml of water and 20 mg of the peptide, would consist of a concentration of 10 mg/ml of the peptide. The afore-mentioned amounts of peptide refer to the net peptide amount (see Example section for a description of net peptide amount).

The sealed container may contain a single dose and thus constitute a unit dose of the peptide, this is usually the case with a pre-filled syringe or syringe cartridge. However, the container may not be emptied in a single injection, for example the same syringe (or cartridge) could deliver multiple injections into a tumour during a single treatment, e.g. 2-8 injections of 0.2 to 0.6 ml per injection. If the sealed container is a vial, it may contain multiple doses, e.g. from 2-12 doses, preferably 2-8 doses.

The sealed container contains a volume of the aqueous formulation of 0.1 to 20.0 ml, preferably 0.2 to 5.0 or 10.0 ml.

In some embodiments, the sealed container is not completely filled with the aqueous formulation of the peptide as defined herein, and the headspace contains air or an inert gas such as nitrogen or argon.

In a further embodiment, the present invention provides a sealed vial or ampoule containing an aqueous formulation of LTX-315, having the amino acid sequence of SEQ ID NO: 23, preferably a pharmaceutically acceptable salt of LTX-315, such as the acetate salt.

It has surprisingly been shown that the medical product can be stored between manufacture and use for at least 1, at least 3, at least 4, at least 6, at least 9, at least 12, at least 18 or at least 24 months, when stored at 8° C. or below, e.g. from 2 to 8° C., such as at around 3, 4, 5, 6, or 7° C. When stored in this way, at least 90%, 91%, 92%, 94%, 95%, 96%, 97%, 98%, 99% or 100% of the peptide concentration is maintained.

Alternatively, the medical product can be stored between manufacture and use for at least 1, at least 2, at least 3, at least 4, at least 5 or at least 6 months, when stored at around 25° C. with up to 60% relative humidity (RH). When stored in this way, at least 90%, 91%, 92%, 93%, 94%, 95%, 96%, 97%, 98%, 99% or 100% of the peptide concentration is maintained.

As shown in the Examples herein, at least 95% of the peptide concentration in a medical product was maintained for at least 24 months, when stored at 8° C. or below, e.g. from 2 to 8° C.

According to the EMA's “ICH Topic Q 1 A (R2) Stability Testing of new Drug Substances and Products”, for drug products intended for storage in a refrigerator, the minimum time period covered by data at submission for long term condition (5° C.±3° C. (i.e. 2-8° C.)) is 12 months, and accelerated condition (25° C.±2° C./60% RH±5% RH) is 6 months. The stability results reported in Examples 1, 2 and 4 support a shelf life of 24 months at 5° C.±3° C. with a potential to propose to the regulatory authorities a shelf life of 36 months by extrapolation of data.

Using the ICH definition of “significant change”, no significant change was observed in any of the tested parameters in the stability study in Examples 1 and 2. In Example 4, using extrapolation from 4 to 6 months, the minor decrease in purity and the corresponding increase in impurities at the storage condition 23±2° C. will not be regarded as a “significant change”. Hence, according to ICH, extrapolation can be used for any proposed shelf life and allowance for the effects of short-term deviations from the label storage conditions, e.g., during shipment and handling, is not required.

### Manufacture of the Medical Product

The medical product of the present invention may be manufactured using any conventional method. The peptide in its solid form, e.g. in the form of a powder, e.g. lyophilised powder, may be dissolved in the aqueous solvent (water, aqueous buffer) under aseptic manufacturing conditions, and the resultant aqueous formulation is preferably sterile filtered and then filled into a container and sealed. The container may be mechanically sealed, e.g. the seal is applied during an industrial process. Alternatively, the peptide may be dissolved in the aqueous solvent (water, aqueous buffer), the resultant aqueous formulation is then filled into a container and sealed. In a preferred embodiment, the container is autoclaved to obtain a sterile aqueous formulation. If excipients are present in the formulation, these are either mixed or dissolved in the aqueous solvent or mixed or dissolved in the resultant aqueous formulation, depending on the nature and amount of the excipient.

Thus, in a further aspect, the present invention provides a method of making a medical product in the form of a sealed container containing an aqueous formulation of the peptide as defined herein, wherein the aqueous formulation is introduced into the container and the filled container is mechanically sealed. In some other embodiments, the invention provides a method of making a medical product in the form of a sealed container containing an aqueous formulation of the peptide as defined herein, wherein the aqueous formulation is prepared as described herein, introduced into the container and the filled container is mechanically sealed. In a preferred embodiment, the aqueous formulation is sterile-filtered before being introduced into the container. In yet another preferred embodiment, the medicinal product is autoclaved.

In some embodiments, the container is not fully filled with the aqueous formulation and the headspace contains air or an inert gas, e.g. nitrogen or argon. Thus, in some embodiments, the invention provides a method of making a medical product in the form of a sealed container containing an aqueous formulation of the peptide as defined herein, wherein the aqueous formulation is introduced into the container without filling it completely and the filled container is mechanically sealed. In some embodiments, the air in the headspace is replaced by an inert gas.

Preferred features of the medical product of the invention discussed above apply, mutatis mutandis, to this aspect of the invention.

Preferred aqueous formulations for use in the medical product of the invention are set out in the Examples.

### Medical Use of the Medical Product

The invention provides methods of treating tumours, both solid and nonsolid tumours, by administering the aqueous formulation contained within the medical product described herein to a subject, e.g. to a subject in need thereof. The amount administered should be a therapeutically effective amount, i.e. effective to kill all or a proportion of the targeted tumour cells or to prevent or reduce their rate of multiplication, or to inhibit metastasis or otherwise to lessen the harmful effect of the tumour on the patient. Such therapeutically effective amount may be administered in one administration, i.e., one dose, or in several administrations, i.e., repetitive doses, i.e., in a series of doses, e.g., over the course of several days, weeks or months and the clinician should observe improvement in one or more of the parameters or symptoms associated with the tumour. The subject will typically be a human patient but non-human animals, such as domestic or livestock animals may also be treated.

Thus, the present invention additionally provides a method of treating a tumour in a subject, comprising obtaining a medical product in the form of a sealed container containing an aqueous formulation of a peptide, or a pharmaceutically acceptable salt thereof, which:
a) consists of 9 amino acids in a linear arrangement;
b) of those 9 amino acids, 5 are cationic and 4 are lipophilic; and
c) of these 4 lipophilic amino acids, 3 are tryptophan and 1 is non-genetically coded and administering the aqueous formulation contained therein to said subject.

In some embodiments, the aqueous formulation is administered into the tumour of the subject, i.e. in some embodiments, the method of treating a tumour in a subject comprises obtaining the medical product of the present invention and administering the aqueous formulation contained therein into the tumour of said subject.

Also disclosed herein is the use of a peptide, or a pharmaceutically acceptable salt thereof, which:
a) consists of 9 amino acids in a linear arrangement;
b) of those 9 amino acids, 5 are cationic and 4 are lipophilic; and
c) of these 4 lipophilic amino acids, 3 are tryptophan and 1 is non-genetically coded
for the manufacture of a medical product in the form of a sealed container containing an aqueous formulation of said peptide or pharmaceutically acceptable salt thereof, for treating a tumour in a subject, wherein the aqueous formulation contained in the sealed container is administered to the subject, e.g. administered into the tumour of said subject.

Also disclosed herein is a medical product in the form of a sealed container containing an aqueous formulation of a peptide, or a pharmaceutically acceptable salt thereof, which:
a) consists of 9 amino acids in a linear arrangement;
b) of those 9 amino acids, 5 are cationic and 4 are lipophilic; and
c) of these 4 lipophilic amino acids, 3 are tryptophan and 1 is non-genetically coded for use in the treatment of a tumour in a subject, wherein the aqueous formulation contained in the sealed container is administered to the subject, e.g. administered into the tumour of said subject.

Cancer targets include carcinomas and adenocarcinomas (particularly from the breast, colon, lung, ovary, pancreas, prostate, skin, kidney and liver (e.g. hepatocellular carcinoma), sarcomas, lymphomas, leukemias, neurological cancer (e.g. from the brain), and melanomas e.g. (skin). Breast, head and neck and skin cancers are preferred targets. Melanoma and carcinoma, are preferred tumour types for treatment. Tumours for treatment are typically solid tumours and may be metastatic lesions that are accessible for transdermal injection.

Preferred features of the medical product of the invention discussed above apply, mutatis mutandis, to these aspects of the invention.

The invention will now be further described with reference to the following non-limiting Examples and the following drawings:

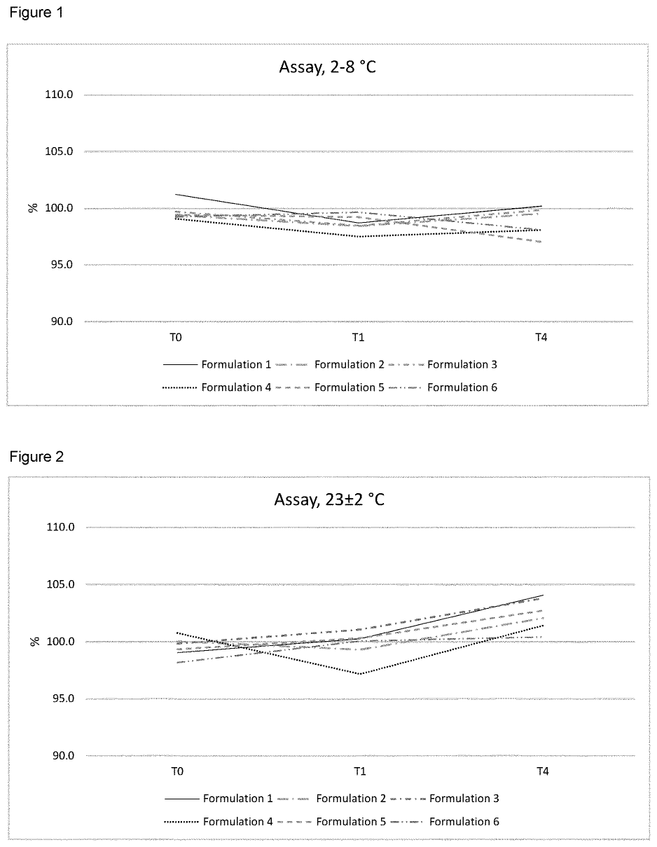

FIG. 1: A graphical presentation of the assay results (%) for every formulation stored at 2-8° C. at T0, T1 and T4 as described in Example 4.

FIG. 2: A graphical presentation of the assay results (%) for every formulation stored at 23±2° C. at T0, T1 and T4 as described in Example 4.

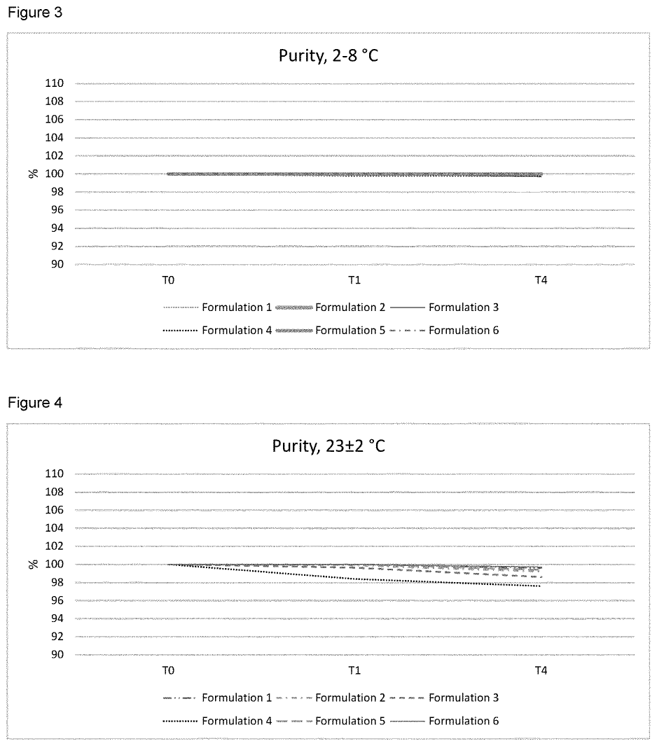

FIG. 3: A graphical presentation of the purity results (%) for every formulation stored at 2-8° C. at T0, T1 and T4 as described in Example 4.

FIG. 4: A graphical presentation of the purity results (%) for every formulation stored at 23±2° C. at T0, T1 and T4 as described in Example 4.

### EXAMPLES

In all of the Examples, the amount of peptide is the net peptide amount. The net peptide content is the fraction of the peptide in question relative to counter-ions, residual water and peptidic impurities. The net peptide amount can be calculated by two different methods:
1. The gross peptide obtained after synthesis is corrected for its purity (determined by HPLC) and the net peptide content (determined by elemental analysis). The net peptide (in %) is calculated as follows:

$$\frac{Net peptide content ( \% ) \times purity ( \% )}{100 \%}$$

The result gives the percentage of net peptide in the gross peptide.
2. The net peptide content corresponds to the assay value calculated from analysis by HPLC with an external standard. The net peptide (in %) is calculated as follows:

$$\frac{Apeptide \times Cs \times 100 \%}{As \times Cpeptide}$$

Where;
Apeptide: peak response of peptide sample
As: peak response of the reference standard
Cpeptide: concentration of peptide sample (mg/ml)
Cs: concentration of reference standard (mg/mL)
The result gives the percentage of net peptide in the gross peptide.

### Example 1.1: Appearance, Osmolality, pH, Colour and Clarity

### Aim

The aim of this study was to determine whether there were any changes to the appearance, osmolality, pH, colour and clarity of the drug product (LTX-315 dissolved in water for injection (WFI)) comprised in sealed vials, after storage for different time periods and under different storage conditions.

### Methods

LTX-315=acetate salt of peptide with sequence K-K-W-W-K-K-W-Dip-K-NH₂ 20 mg/mL LTX-315 drug product was prepared by dissolving LTX-315 powder in WFI and filtered through a 0.22 μm sterile filter. 1 ml of the drug product was then filled into vials and sealed. The WFI complied to the Ph. Eur monograph 0169 for WFI. All product contact vials, glassware and peripherals were sterilised by depyrogenation in a hot air oven at 250° C. for 120 minutes after twice rinsing with WFI. Product contact tubing, stoppers and over-seals were twice rinsed with WFI then sterilised by autoclaving for >15 minutes at 121° C. then dried overnight in a drying oven. The vials were stored as set out in Table 1 below and analysed according to the listed parameters. Appearance was tested visually according to Ph.Eur 2.9.20 and osmolality according to Ph. Eur. 2.2.35. pH was tested according to Ph. Eur. 2.2.3. Colour and clarity were tested according to Ph. Eur 2.2.2 and Ph. Eur 2.2.1, respectively.

### Results and Conclusions

The drug product was deemed to be of satisfactory appearance, osmolality pH, colour and clarity, at T=0. There was no change in any of these parameters after 24 months' storage in the dark in a refrigerator at 2-8° C. or 6 months at 25° C. with 60% relative humidity (RH) in the dark in a climate chamber.

TABLE 1
Appearance, Osmolality, pH, Colour and Clarity of solution

| Time-point/ | | | Osmolality | | Col- | |
|---|---|---|---|---|---|---|
| storage | | Appearance | mOsmkg⁻¹ | pH | our | Clarity |
| Initial (T = 0) | | A clear, | 132 | 6.1 | >B9 | Turbidity |
| | | colourless | | | | comparable |
| | | solution | | | | with |
| | | free from | | | | 1 NTU |
| | | visible | | | | standard |
| | | particulates | | | | solution |
| 2-8° C. | T = 1 | A clear, | — | | >B9 | Turbidity |
| | month | colourless | | | | comparable |
| | | solution | | | | with |
| | | free from | | | | 1 NTU |
| | | visible | | | | standard |
| | | particulates | | | | solution |
| | T = 3 | A clear, | — | 6.2 | >B9 | Turbidity |
| | months | colourless | | | | comparable |
| | | solution | | | | with |
| | | free from | | | | 1 NTU |
| | | visible | | | | standard |
| | | particulates | | | | solution |
| | T = 6 | A clear, | 133 | 6.0 | >B9 | Turbidity |
| | months | colourless | | | | comparable |
| | | solution | | | | with |
| | | free from | | | | 1 NTU |
| | | visible | | | | standard |
| | | particulates | | | | solution |
| | T = 9 | A clear, | — | | >B9 | Turbidity |
| | months | colourless | | | | comparable |
| | | solution | | | | with |
| | | free from | | | | 1 NTU |
| | | visible | | | | standard |
| | | particulates | | | | solution |
| | T = 12 | A clear, | 133 | 6.1 | >B9 | Turbidity |
| | months | colourless | | | | comparable |
| | | solution | | | | with |
| | | free from | | | | 1 NTU |
| | | visible | | | | standard |
| | | particulates | | | | solution |
| | T = 18 | A clear, | — | | >B9 | Turbidity |
| | months | colourless | | | | comparable |
| | | solution | | | | with |
| | | free from | | | | 1 NTU |
| | | visible | | | | standard |
| | | particulates | | | | solution |
| | T = 24 | A clear, | 132 | 6.1 | >B9 | Turbidity |
| | months | colourless | | | | comparable |
| | | solution | | | | with |
| | | free from | | | | 1 NTU |
| | | visible | | | | standard |
| | | particulates | | | | solution |
| 25° C./ | T = 1 | A clear, | — | | >B9 | Turbidity |
| 60% | month | colourless | | | | comparable |
| RH | | solution | | | | with |
| | | free from | | | | 1 NTU |
| | | visible | | | | standard |
| | | particulates | | | | solution |
| | T = 3 | A clear, | — | 6.2 | >B9 | Turbidity |
| | months | colourless | | | | comparable |
| | | solution | | | | with |
| | | free from | | | | 1 NTU |
| | | visible | | | | standard |
| | | particulates | | | | solution |
| | T = 6 | A clear, | 133 | 6.1 | >B9 | Turbidity |
| | months | colourless | | | | comparable |
| | | solution | | | | with 1 NTU |
| | | free from | | | | standard |
| | | visible | | | | solution |
| | | particulates | | | | |

B9 and 1 NTU are reference standards described in Ph. Eur. 2.2.1 and Ph. Eur. 2.2.2

### Example 1.2: LTX-315 Concentration

### Aim

The aim of this study was to determine whether there were any changes to the concentration of LTX-315 in the drug product (LTX-315 dissolved in WFI) comprised in sealed vials after storage for different time periods and under different storage conditions.

### Methods

Drug product was prepared and stored as described in Example 1.1.

Reversed phase HPLC, using a trifluoracetic acid (TFA) system and UV detection at 220 nm, was used to determine the amount of LTX-315 after storage of the vials in accordance with the storage conditions set out in Table 2.

The column used was an RP C18, e.g. Waters Sunfire, 3.5 μm, 4.6×150 mm. The LTX-315 concentration in the test sample solution was 0.5 mg/ml and the injection volume 10 μl. The gradient profile used is set out below and the flow rate was 1.2 mL/min.

Gradient profile

| | | | Effective content of |
|---|---|---|---|
| Time [min] | Eluent A [%] | Eluent B [%] | ACN [%] |
| 0 | 85 | 15 | 15.9 |
| 3 | 85 | 15 | 15.9 |
| 15 | 75 | 25 | 25.8 |
| 25 | 70 | 30 | 30.7 |
| 28 | 70 | 30 | 30.7 |
| 28.1 | 85 | 15 | 15.9 |
| 33 | 85 | 15 | 15.9 |

Eluent A: Into a 2 L volumetric flask was added approx. 1800 mL of water, 20 mL of ACN (acetonitrile), and 2 mL of TFA. Water was added to volume and mixed well.
Eluent B: Into a 2 L volumetric flask was added approx. 1800 mL of ACN, and 2 mL of TFA. ACN was added to volume and mixed well.

### Results and Conclusions

The concentration of LTX-315 after storage at 25° C. with 60% relative humidity for six months is the same as the concentration at T=0. The slight variations in concentration are due to the variability in the analytical method. There is a very slight decrease in the LTX-315 concentration after storage at 2-8° C. for 24 months. These results indicate that LTX-315 is stable in aqueous solution at these storage conditions for this time period.

TABLE 2

| | | | | Mean | Rec./ | Rec./ |
|---|---|---|---|---|---|---|
| | | Vial 1 | Vial 2 | content | theory¹ | T = 0² |
| Time-point/storage | | (mg/mL) | (mg/mL) | (mg/mL) | (%) | (%) |
| Initial (T = 0) | | 21.068 | 20.978 | 21.0 | 105.1 | — |
| 2-8° C. | T = 1 month | 20.548 | 19.916 | 20.2 | 101.2 | 96.3 |
| | T = 3 months | 21.241 | 21.276 | 21.2 | 106.3 | 101.1 |
| | T = 6 months | 20.979 | 21.027 | 21.0 | 105.0 | 99.9 |
| | T = 9 months | 20.311 | 20.631 | 20.4 | 102.4 | 97.4 |
| | T = 12 months | 20.895 | 20.631 | 20.7 | 103.8 | 98.8 |
| | T = 18 months | 19.204 | 19.990 | 19.6 | 98.0 | 93.2 |
| | T = 24 months | 20.150 | 19.669 | 19.9 | 99.5 | 94.7 |
| 25° C./ | T = 1 month | 20.269 | 20.194 | 20.2 | 101.2 | 96.2 |
| 60% RH | T = 3 months | 20.897 | 20.222 | 20.5 | 102.8 | 97.8 |
| | T = 6 months | 20.575 | 20.215 | 20.3 | 102.0 | 97.0 |

¹Calculated as percentage of theoretical 20 mg/mL
²% Recovery vs pre-filtration results

### Example 1.3: Purity

### Aim

The aim of this study was to determine whether there were any changes to the purity of the drug product (LTX-315 dissolved in WFI) comprised in sealed vials after storage for different time periods and under different storage conditions.

### Materials and Methods

Drug product was prepared and stored as described in Example 1.1.

HPLC analysis was performed as described in Example 1.2 to determine the concentration of LTX-315 and the amount of related substances, i.e. substances formed due to and thus related to the degradation of LTX-315 in the drug product.

### Results and Conclusions

There is a very slight increase in total related substances observed for drug product stored at 2-8° C. from 0.88% at T=0 to 1.64% after 24 months, as shown in Table 3. There is also a slight increase in total related substances observed for drug product stored at 25° C./60% RH, from 0.88% at T=0 to 2.31% at T=6 months, as shown in Table 4.

TABLE 3
Purity/related substances (area %), 2-8° C.

| | Time-point and amount (area %) | | | | | | | |
|---|---|---|---|---|---|---|---|---|
| | Initial | | | | | | | |
| RRT | (T = 0) | 1 M | 3 M | 6 M | 9 M | 12 M | 18 M | 24 M |
| 0.62 | ND | ND | ND | ND | ND | ND | ND | <LOQ |
| 0.75 | <LOQ | <LOQ | <LOQ | <LOQ | ND | <LOQ | <LOQ | <LOQ |
| 0.80 | ND | ND | ND | ND | ND | ND | 0.05 | <LOQ |
| 0.86 | ND | ND | ND | ND | 0.07 | <LOQ | 0.05 | 0.05 |
| 0.88 | <LOQ | <LOQ | <LOQ | ND | ND | <LOQ | <LOQ | <LOQ |
| 0.89 | ND | ND | ND | ND | 0.07 | 0.05 | <LOQ | <LOQ |
| 0.90 | <LOQ | <LOQ | <LOQ | ND | <LOQ | <LOQ | <LOQ | <LOQ |
| 0.908 | ND | ND | ND | ND | ND | ND | 0.06 | 0.05 |
| 0.91 | <LOQ | <LOQ | <LOQ | <LOQ | 0.07 | 0.07 | 0.08 | 0.10 |
| 0.92 | ND | ND | ND | ND | 0.07 | 0.06 | 0.08 | 0.08 |
| 0.93 | ND | ND | ND | ND | ND | ND | <LOQ | ND |
| 0.94 | ND | ND | ND | ND | 0.06 | 0.05 | 0.15 | 0.05 |
| 0.95 | 0.08 | 0.09 | 0.07 | 0.07 | 0.0707 | 0.05 | 0.06 | 0.05 |
| 0.96 | <LOQ | 0.06 | <LOQ | <LOQ | 0.10 | 0.09 | <LOQ | 0.10 |
| 0.98 | 0.10 | 0.06 | 0.05 | 0.08 | 0.11 | 0.10 | 0.05 | 0.09 |
| 1.02 | 0.32 | 0.41 | 0.39 | 0.31 | 0.39 | 0.33 | 0.26 | 0.41 |
| 1.04 | <LOQ | ND | 0.10 | 0.05 | 0.07 | 0.07 | 0.07 | 0.10 |
| 1.05 | 0.18 | 0.28 | 0.23 | 0.17 | 0.18 | 0.17 | 0.20 | 0.19 |
| 1.055 | 0.13 | 0.19 | 0.23 | 0.16 | 0.18 | 0.18 | 0.13 | 0.22 |
| 1.06 | ND | ND | 0.05 | <LOQ | ND | ND | ND | ND |
| 1.07 | <LOQ | 0.08 | 0.09 | <LOQ | 0.05 | 0.05 | ND | ND |
| 1.09 | <LOQ | 0.07 | 0.05 | 0.06 | <LOQ | <LOQ | 0.08 | 0.08 |
| 1.094 | ND | ND | ND | ND | ND | ND | <LOQ | <LOQ |
| 1.13 | <LOQ | <LOQ | <LOQ | ND | ND | ND | <LOQ | <LOQ |
| 1.14 | <LOQ | <LOQ | <LOQ | ND | ND | ND | <LOQ | <LOQ |
| 1.16 | <LOQ | <LOQ | <LOQ | ND | ND | ND | <LOQ | <LOQ |
| 1.19 | <LOQ | <LOQ | <LOQ | ND | <LOQ | <LOQ | ND | <LOQ |
| 1.21 | 0.07 | 0.06 | 0.06 | 0.08 | 0.06 | 0.06 | 0.06 | 0.06 |
| Total¹ | 0.88 | 1.31 | 1.32 | 0.98 | 1.54 | 1.34 | 1.37 | 1.64 |

¹Sum of related substances >0.05%
LOQ = limit of quantification

TABLE 4
Purity/related substances (area %), 25° C./60% RH

Time-point and amount (area %)

| RRT | Initial (T = 0) | 1 M | 3 M | 6 M |
|---|---|---|---|---|
| 0.75 | <LOQ | <LOQ | 0.05 | <LOQ |
| 0.79 | ND | ND | ND | 0.05 |
| 0.80 | ND | ND | ND | 0.06 |
| 0.88 | <LOQ | <LOQ | <LOQ | 0.05 |
| 0.90 | <LOQ | <LOQ | 0.05 | 0.05 |
| 0.91 | <LOQ | 0.06 | 0.05 | 0.08 |
| 0.945 | <LOQ | 0.07 | <LOQ | 0.09 |
| 0.95 | <LOQ | 0.05 | <LOQ | 0.05 |
| 0.955 | 0.08 | 0.08 | 0.12 | 0.10 |
| 0.96 | <LOQ | <LOQ | <LOQ | <LOQ |
| 0.98 | 0.10 | 0.07 | 0.07 | 0.05 |
| 1.02 | 0.32 | 0.42 | 0.43 | 0.35 |
| 1.04 | <LOQ | 0.10 | 0.25 | 0.34 |
| 1.05 | 0.18 | 0.22 | 0.19 | 0.15 |
| 1.055 | 0.13 | 0.25 | 0.34 | 0.37 |
| 1.07 | <LOQ | 0.09 | 0.07 | 0.05 |
| 1.09 | <LOQ | 0.09 | 0.28 | 0.39 |
| 1.13 | <LOQ | <LOQ | <LOQ | ND |
| 1.14 | <LOQ | <LOQ | <LOQ | ND |
| 1.16 | <LOQ | <LOQ | <LOQ | ND |
| 1.19 | <LOQ | <LOQ | <LOQ | ND |
| 1.21 | 0.07 | 0.06 | 0.07 | 0.10 |
| Total¹ | 0.88 | 1.55 | 1.97 | 2.31 |

¹Sum of related substances >0.05%
LOQ = limit of quantification

### Conclusions

The results in Example 1.1, 1.2 and 1.3 show that an aqueous formulation of LTX-315 as an aqueous solution dissolved in WFI and comprised in a sealed container is stable; there is no change in appearance, osmolality, pH, colour or clarity for up to 24 months when the sealed container is stored at 2-8° C. or for up to 6 months at 25° C./60% RH. For a medical product, the concentration of the active ingredient (here LTX-315) must not be less than 90% of the concentration during the shelf life of the product. As shown in Example 1.2, very few changes were determined in LTX-315 concentration, which is also reflected by the minor changes in the related substances, as shown in Example 1.3, at these storage conditions and time period.

### Example 2: Impact of pH

### Aim

The aim of this study was to investigate the effect of pH on the stability of an aqueous formulation of LTX-315 in WFI comprised in a sealed container.

### Materials and Methods

The stability of solutions of LTX-315 dissolved in WFI at a concentration of 20 mg/mL were assessed at pH 5, pH 6, pH 7 and pH 8. pH adjustment was achieved by using dilute acetic acid (10%) and aqueous sodium hydroxide solution (0.1M, 0.5M and 1M NaOH). The formulation details are shown in Table 5 and the testing matrix in Table 6.

The samples were prepared by dissolving LTX-315 in WFI, each solution was stirred for 30 minutes to mix and then the solutions were adjusted to the target pH using NaOH and/or acetic acid. Once the target pH had been achieved, each solution was transferred to individual 25 mL volumetric flasks and made to volume with WFI. The pH at final volume was measured and all solutions were still at target. Each solution was then passed through a 0.2 μm PES membrane syringe filter. The pH of the filtered solutions was measured; all solutions were still at target pH. Each solution was filled at a volume of 1 mL into clear pharmaceutical type 1 glass 2 mL vials, stoppered and sealed.

The samples were incubated at a single stress condition (40° C./75% RH). The vial contents were then assessed for appearance, pH, LTX-315 concentration and purity at T=0 and then for stability at four additional times-points (2, 5, 7 and 14 days). Additional vials were held at ambient, light-exposed laboratory conditions and tested along with the stress condition. Control solutions were held at 2-8° C. for analysis at the final time point only. No changes in appearance and pH were observed for the control solutions.

Appearance was assessed by visual examination as per Ph. Eur. 2.9.20; pH was measured as per Ph. Eur. 2.2.3 and concentration, as well as levels of related substances, were assessed by HPLC as described above (Example 1.2).

TABLE 5
Formulations

| | LTX-315 concentration | |
|---|---|---|
| Solution | (mg/mL) | pH |
| LYT-01 | 20 | 5 |
| LYT-02 | 20 | 6 |
| LYT-03 | 20 | 7 |
| LYT-04 | 20 | 8 |

TABLE 6
Testing matrix

| | | | | | T = 14 days |
|---|---|---|---|---|---|
| | | T = 2 days | T = 5 days | T = 7 days | (2-8° C., |
| | | (ambient | (ambient | (ambient | ambient |
| Testing | T = 0 | and stress) | and stress) | and stress) | and stress |
| pH | X | X | X | X | X |
| Concentration/ | X | X | X | X | X |
| RS | | | | | |
| Appearance | X | X | X | X | X |

RS: related substances

### Results

### Appearance and pH

There was no significant change in the appearance or pH of each formulation regardless of pH at both ambient and stress (40° C./75% RH) storage condition up to 14 days (see Table 7-10).

TABLE 7
Appearance and pH, LYT-01

| Time-point/storage | | Appearance | pH |
|---|---|---|---|
| Initial (T = 0) | | Clear, colourless solution with no visible particulates | 5.1 |
| Ambient | T = 2 days | Clear, colourless solution with no visible particulates | 5.1 |
| | T = 5 days | Clear, colourless solution with no visible particulates | 5.1 |
| | T = 7 days | Clear, colourless solution with no visible particulates | 5.1 |
| | T = 14 days | Clear, colourless solution with no visible particulates | 5.3 |
| 40° C./75% RH | T = 2 days | Clear, colourless solution with no visible particulates | 5.1 |
| | T = 5 days | Clear, colourless solution with no visible particulates | 5.0 |
| | T = 7 days | Clear, colourless solution with no visible particulates | 5.1 |
| | T = 14 days | Clear, colourless solution with no visible particulates | 5.2 |

TABLE 8
Appearance and pH, LYT-02

| Time point/storage | | Appearance | pH |
|---|---|---|---|
| Initial (T = 0) | | Clear, colourless solution with no visible particulates | 6.1 |
| Ambient | T = 2 days | Clear, colourless solution with no visible particulates | 6.1 |
| | T = 5 days | Clear, colourless solution with no visible particulates | 6.1 |
| | T = 7 days | Clear, colourless solution with no visible particulates | 6.0 |
| | T = 14 days | Clear, colourless solution with no visible particulates | 6.2 |
| 40° C./75% RH | T = 2 days | Clear, colourless solution with no visible particulates | 6.1 |
| | T = 5 days | Clear, colourless solution with no visible particulates | 6.0 |
| | T = 7 days | Clear, colourless solution with no visible particulates | 6.0 |
| | T = 14 days | Clear, colourless solution with no visible particulates | 6.2 |

TABLE 9
Appearance and pH, LYT-03

| Time-point/storage | | Appearance | pH |
|---|---|---|---|
| Initial (T = 0) | | Clear, colourless solution with no visible particulates | 7.1 |
| Ambient | T = 2 days | Clear, colourless solution with no visible particulates | 7.1 |
| | T = 5 days | Clear, colourless solution with no visible particulates | 7.1 |
| | T = 7 days | Clear, colourless solution with no visible particulates | 7.1 |
| | T = 14 days | Clear, colourless solution with no visible particulates | 7.2 |
| 40° C./75% RH | T = 2 days | Clear, colourless solution with no visible particulates | 7.5 |
| | T = 5 days | Clear, colourless solution with no visible particulates | 7.1 |
| | T = 7 days | Clear, colourless solution with no visible particulates | 7.1 |
| | T = 14 days | Clear, colourless solution with no visible particulates | 7.2 |

TABLE 10
Appearance and pH, LYT-04

| Time-point/storage | | Appearance | pH |
|---|---|---|---|
| Initial (T = 0) | | Clear, colourless solution with no visible particulates | 8.1 |
| Ambient | T = 2 days | Clear, colourless solution with no visible particulates | 8.0 |
| | T = 5 days | Clear, colourless solution with no visible particulates | 8.1 |
| | T = 7 days | Clear, colourless solution with no visible particulates | 8.0 |
| | T = 14 days | Clear, colourless solution with no visible particulates | 8.1 |
| 40° C./75% RH | T = 2 days | Clear, colourless solution with no visible particulates | 8.0 |
| | T = 5 days | Clear, colourless solution with no visible particulates | 8.0 |
| | T = 7 days | Clear, colourless solution with no visible particulates | 8.0 |
| | T = 14 days | Clear, colourless solution with no visible particulates | 8.0 |

### Concentration

The effect of pH on the stability of the various LTX-315 formulations was determined by measuring the concentration of LTX-315 (assay) and the results are shown in Tables 11-15. The initial assay suggests that LTX-315 concentration in all formulations is slightly higher than the target concentration of 20 mg/ml (102.2-103.6%). There is no significant change in concentration of each of formulations LYT-01, LYT-02 and LYT-03 (pH 5, 6 and 7, respectively over the 14-day period at both ambient and stress conditions. For formulation LYT-04 (pH 8), there is no change in concentration observed at ambient storage condition, whereas a small reduction in concentration was seen at the 40° C./75% RH stress condition.

TABLE 11
Assay, LYT-01

| | | | | | Rec./ | Rec/ | Rec./ |
|---|---|---|---|---|---|---|---|
| | | Vial 1 | Vial 2 | Mean | theory¹ | prefilter² | T = 0³ |
| Time-point/storage | | (mg/mL) | (mg/mL) | (mg/mL) | (%) | (%) | (%) |
| Pre-filter | | 20.846 | 20.893 | 20.87 | 104.3 | — | — |
| Initial (T = 0) | | 20.867 | 20.592 | 20.73 | 103.6 | 99.3 | — |
| Ambient | T = 2 days | 20.896 | 20.745 | 20.82 | 104.1 | | 100.4 |
| | T = 5 days | 20.741 | 20.708 | 20.72 | 103.6 | | 100.0 |
| | T = 7 days | 20.957 | 21.108 | 21.03 | 105.2 | | 101.5 |
| | T = 14 days | 21.079 | 21.114 | 21.10 | 105.5 | | 101.8 |
| 40° C./ | T = 2 days | 21.448 | 20.926 | 21.19 | 105.9 | | 102.2 |
| 75% RH | T = 5 days | 20.846 | 20.973 | 20.91 | 104.5 | | 100.9 |
| | T = 7 days | 21.029 | 20.999 | 21.01 | 105.1 | | 101.4 |
| | T = 14 days | 21.081 | 20.982 | 21.03 | 105.2 | | 101.5 |

¹As percentage of theoretical 20 mg/mL
²As percentage of theoretical of pre-filtration assay
³As percentage of the initial (T = 0) result.

TABLE 12
Assay, LYT-02

| | | | | | Rec./ | Rec/ | Rec./ |
|---|---|---|---|---|---|---|---|
| | | Vial 1 | Vial 2 | Mean | theory ¹ | prefilter² | T = 0³ |
| Time-point/storage | | mg/mL) | (mg/mL) | (mg/mL) | (%) | (%) | (%) |
| Pre-filter | | 20.670 | 20.655 | 20.66 | 103.3 | — | |
| Initial (T = 0) | | 20.646 | 20.807 | 20.73 | 103.6 | 100.3 | |
| Ambient | T = 2 days | 20.792 | 20.583 | 20.69 | 103.4 | | 99.8 |
| | T = 5 days | 20.576 | 20.698 | 20.64 | 103.2 | | 99.6 |
| | T = 7 days | 20.929 | 20.954 | 20.94 | 104.7 | | 101.0 |
| | T = 14 days | 20.720 | 20.577 | 20.65 | 103.2 | | 99.6 |
| 40° C./ | T = 2 days | 20.930 | 21.041 | 20.99 | 104.9 | | 101.2 |
| 75% RH | T = 5 days | 20.745 | 20.8(3 | 20.78 | 103.9 | | 100.2 |
| | T = 7 days | 20.879 | 20.857 | 20.87 | 104.3 | | 100.7 |
| | T = 14 days | 20.935 | 21.022 | 20.98 | 104.9 | | 101.2 |

¹ As percentage of theoretical 20 mg/mL
²As percentage of theoretical of pre-filtration assay
³As percentage of the initial (T = 0) result.

TABLE 13
Assay, LYT-03

| | | | | | Rec./ | Rec/ | Rec./ |
|---|---|---|---|---|---|---|---|
| | | Vial 1 | Vial 2 | Mean | theory¹ | prefilter² | T = 0³ |
| Time-point/storage | | (mg/mL) | (mg/mL) | (mg/mL) | (%) | (%) | (%) |
| Pre-filter | | 20.501 | 20.755 | 20.63 | 103.1 | — | — |
| Initial (T = 0) | | 20.712 | 20.707 | 20.71 | 103.5 | 100.4 | — |
| Ambient | T = 2 | 20.742 | 20.816 | 20.78 | 103.9 | | 100.3 |
| | days | | | | | | |
| | T = 5 | 20.930 | 20.496 | 20.71 | 103.6 | | 100.0 |
| | days | | | | | | |
| | T = 7 | 20.984 | 20.919 | 20.95 | 104.8 | | 101.2 |
| | days | | | | | | |
| | T = 14 | 20.808 | 20.928 | 20.87 | 104.3 | | 100.8 |
| | days | | | | | | |
| 40° C./ | T = 2 | 20.736 | 20.656 | 20.70 | 103.5 | | 99.9 |
| 75% RH | days | | | | | | |
| | T = 5 | 21.091 | 20.617 | 20.85 | 104.3 | | 100.7 |
| | days | | | | | | |
| | T = 7 | 20.787 | 20.767 | 20.78 | 103.9 | | 100.3 |
| | days | | | | | | |
| | T = 14 | 20./90 | 20.688 | 20.74 | 103.7 | | 100.1 |
| | days | | | | | | |

¹As percentage of theoretical 20 mg/mL
²As percentage of theoretical of pre-filtration assay
³As percentage of the initial (T = 0) result.

TABLE 14
Assay, LYT-04

| | | | | | Rec./ | Rec/ | Rec./ |
|---|---|---|---|---|---|---|---|
| | | Vial 1 | Vial 2 | Mean | theory¹ | prefilter² | T = 0³ |
| Time-point/storage | | (mg/mL) | (mg/mL) | (mg/mL) | (%) | (%) | (%) |
| Pre-filter | | 20.239 | 20.366 | 20.30 | 101.5 | — | — |
| Initial (T = 0) | | 20.531 | 20.336 | 20.43 | 102.2 | 100.6 | |
| Ambient | T = 2 | 20.448 | 21.311 | 20.88 | 104.4 | | 102.2 |
| | days | | | | | | |
| | T = 5 | 20.769 | 20.369 | 20.57 | 102.8 | | 100.7 |
| | days | | | | | | |
| | T = 7 | 20.710 | 20.633 | 20.67 | 103.4 | | 101.2 |
| | days | | | | | | |
| | T = 14 | 21.040 | 20.962 | 21.00 | 105.0 | | 102.8 |
| | days | | | | | | |
| 40° C./ | T = 2 | 20.678 | 20.758 | 20.72 | 103.6 | | 101.4 |
| 75% RH | days | | | | | | |
| | T = 5 | 20.331 | 20.386 | 20.36 | 101.8 | | 99.6 |
| | days | | | | | | |
| | T = 7 | 20.336 | 20.372 | 20.35 | 101.8 | | 99.6 |
| | days | | | | | | |
| | T = 14 | 20.100 | 20.090 | 20.10 | 100.5 | | 98.4 |
| | days | | | | | | |

¹As percentage of theoretical 20 mg/mL
²As percentage of theoretical of pre-filtration assay
³As percentage of the initial (T = 0) result.

### Purity/Related Substances

There was no significant increase in total related substances for formulations LYT-01 (pH 5) and LYT-02 (pH 6) observed, when stored at ambient or 40° C./75% RH conditions for 14-days (Tables 15-18). There was a modest increase in total related substances for batch LYT-03 (pH 7) when stored at ambient and especially at 40° C./75% RH conditions over the 14-day storage (Tables 19 and 20).

There was an increase in total related substances for LYT-04 (pH 8) when stored at 40° C./75% RH conditions over the 14-day storage (Tables 21 and 22).

TABLE 15
Purity/related substances (area %), LYT-01 ambient

| | Time-point and amount (area %) | | | | | |
|---|---|---|---|---|---|---|
| | Pre- | | T = 2 | T = 5 | T = 7 | T = 14 |
| | filtration | T = 0 | days | days | days | days |
| RRT 0.75 | <LOQ | <LOQ | <LOQ | <LOQ | <LOQ | <LOQ |
| RRT 0.88 | <LOQ | <LOQ | <LOQ | <LOQ | <LOQ | <LOQ |
| RRT 0.89 | <LOQ | <LOQ | <LOQ | <LOQ | <LOQ | <LOQ |
| RRT 0.90 | <LOQ | <LOQ | 0.05 | <LOQ | <LOQ | <LOQ |
| RRT 0.91 | 0.05 | <LOQ | <LOQ | 0.07 | 0.05 | <LOQ |
| RRT 0.94 | <LOQ | <LOQ | <LOQ | <LOQ | <LOQ | <LOQ |
| RRT 0.95 | <LOQ | <LOQ | 0.06 | <LOQ | <LOQ | <LOQ |
| RRT 0.955 | <LOQ | <LOQ | IND | <LOQ | <LOQ | <LOQ |
| RRT 0.96 | 0.09 | 0.09 | 0.10 | 0.08 | 0.09 | 0.08 |
| RRT 0.98 | 0.10 | 0.09 | 0.10 | 0.09 | 0.10 | 0.08 |
| LTX-315 | 98.42 | 98.46 | 98.44 | 98.56 | 98.43 | 98.72 |
| RRT 1.02 | 0.40 | 0.40 | 0.37 | 0.41 | 0.41 | 0.40 |
| RRT 1.04 | <LOQ | <LOQ | 0.06 | 0.05 | 0.07 | 0.06 |
| RRT 1.05 | 0.28 | 0.28 | 0.22 | 0.20 | 0.21 | 0.19 |
| RRT 1.055 | 0.16 | 0.16 | 0.16 | 0.14 | 0.16 | 0.13 |
| RRT 1.06 | <LOQ | <LOQ | <LOQ | <LOQ | <LOQ | <LOQ |
| RRT 1.07 | 0.07 | 0.06 | 0.11 | 0.05 | 0.07 | <LOQ |
| RRT 1.09 | <LOQ | <LOQ | <LOQ | <LOQ | <LOQ | 0.05 |
| RRT 1.10 | <LOQ | <LOQ | <LOQ | <LOQ | <LOQ | <LOQ |
| RRT 1.11 | <LOQ | <LOQ | <LOQ | <LOQ | <LOQ | ND |
| RRT 1.12 | <LOQ | <LOQ | <LOQ | ND | ND | ND |
| RRT 1.14 | <LOQ | <LOQ | <LOQ | <LOQ | <LOQ | <LOQ |
| RRT 1.16 | <LOQ | <LOQ | <LOQ | <LOQ | <LOQ | <LOQ |
| RRT 1.17 | <LOQ | <LOQ | <LOQ | IND | ND | ND |
| RRT 1.19 | <LOQ | <LOQ | <LOQ | <LOQ | <LOQ | <LOQ |
| RRT 1.21 | 0.07 | 0.07 | 0.06 | 0.06 | 0.06 | 0.05 |
| Total¹ | 1.23 | 1.17 | 1.29 | 1.16 | 1.21 | 1.03 |

¹Sum of related substances >0.05%
ND = Not detected
LOQ = limit of quantification

TABLE 16
Purity/related substances (area %), LYT-01 40° C./75% RH

| | Time-point and amount (area %) | | | | | |
|---|---|---|---|---|---|---|
| | Pre- | | T = 2 | T = 5 | T = 7 | T = 14 |
| RRT | filtration | T = 0 | days | days | days | days |
| RRT 0.75 | <LOQ | <LOQ | <LOQ | <LOQ | <LOQ | <LOQ |
| RRT 0.88 | <LOQ | <LOQ | <LOQ | 0.05 | <LOQ | <LOQ |
| RRT 0.89 | <LOQ | <LOQ | <LOQ | <LOQ | <LOQ | <LOQ |
| RRT 0.90 | <LOQ | <LOQ | <LOQ | <LOQ | <LOQ | <LOQ |
| RRT 0.91 | 0.05 | <LOQ | <LOQ | <LOQ | 0.07 | 0.06 |
| RRT 0.94 | <LOQ | <LOQ | <LOQ | <LOQ | <LOQ | <LOQ |
| RRT 0.95 | <LOQ | <LOQ | 0.09 | 0.05 | <LOQ | <LOQ |
| RRT 0.955 | <LOQ | <LOQ | ND | <LOQ | <LOQ | 0.06 |
| RRT 0.96 | 0.09 | 0.09 | 0.14 | 0.10 | 0.09 | 0.10 |
| RRT 0.98 | 0.10 | 0.09 | 0.12 | 0.12 | 0.11 | 0.09 |
| LTX-315 | 98.42 | 98.46 | 98.11 | 98.15 | 98.28 | 98.16 |
| RRT 1.02 | 0.40 | 0.40 | 0.38 | 0.44 | 0.40 | 0.45 |
| RRT 1.04 | <LOQ | <LOQ | 0.08 | 0.08 | 0.10 | 0.14 |
| RRT 1.05 | 0.28 | 0.28 | 0.22 | 0.21 | 0.22 | 0.19 |
| RRT 1.055 | 0.16 | 0.16 | 0.18 | 0.16 | 0.16 | 0.15 |
| RRT 1.06 | <LOQ | <LOQ | 0.05 | <LOQ | <LOQ | ND |
| RRT 1.07 | 0.07 | 0.06 | 0.12 | 0.07 | 0.06 | <LOQ |
| RRT 1.09 | <LOQ | <LOQ | 0.09 | 0.11 | 0.13 | 0.24 |
| RRT 1.10 | <LOQ | <LOQ | <LOQ | <LOQ | <LOQ | <LOQ |
| RRT 1.11 | <LOQ | <LOQ | <LOQ | <LOQ | ND | <LOQ |
| RRT 1.12 | <LOQ | <LOQ | ND | ND | ND | ND |
| RRT 1.14 | <LOQ | <LOQ | <LOQ | <LOQ | <LOQ | <LOQ |
| RRT 1.16 | <LOQ | <LOQ | <LOQ | <LOQ | <LOQ | <LOQ |
| RRT 1.17 | <LOQ | <LOQ | ND | ND | <LOQ | <LOQ |
| RRT 1.19 | <LOQ | <LOQ | <LOQ | <LOQ | <LOQ | <LOQ |
| RRT 1.21 | 0.07 | 0.07 | 0.06 | 0.06 | 0.07 | 0.06 |
| Total¹ | 1.23 | 1.17 | 1.54 | 1.40 | 1.40 | 1.40 |

¹Sum of related substances >0.05%
ND = Not detected
LOQ = limit of quantification

TABLE 17
Purity/related substances (area %), LYT-02 ambient

| | Time-point and amount (area %) | | | | | |
|---|---|---|---|---|---|---|
| | Pre- | | T = 2 | T = 5 | T = 7 | T = 14 |
| RRT | filtration | T = 0 | days | days | days | days |
| RRT 0.75 | 0.05 | <LOQ | <LOQ | <LOQ | <LOQ | <LOQ |
| RRT 0.88 | <LOQ | <LOQ | <LOQ | <LOQ | <LOQ | <LOQ |
| RRT 0.89 | <LOQ | <LOQ | <LOQ | <LOQ | <LOQ | <LOQ |
| RRT 0.90 | <LOQ | <LOQ | <LOQ | <LOQ | <LOQ | <LOQ |
| RRT 0.91 | 0.07 | 0.06 | <LOQ | <LOQ | 0.07 | 0.05 |
| RRT 0.94 | <LOQ | <LOQ | <LOQ | <LOQ | <LOQ | <LOQ |
| RRT 0.95 | <LOQ | <LOQ | 0.07 | <LOQ | <LOQ | <LOQ |
| RRT 0.955 | <LOQ | <LOQ | ND | <LOQ | <LOQ | 0.13 |
| RRT 0.96 | 0.08 | 0.09 | 0.11 | 0.09 | 0.09 | 0.11 |
| RRT 0.98 | 0.10 | 0.09 | 0.11 | 0.11 | 0.11 | 0.09 |
| LTX-315 | 98.40 | 98.53 | 98.33 | 98.57 | 98.45 | 98.46 |
| RRT 1.02 | 0.41 | 0.39 | 0.39 | 0.40 | 0.41 | 0.48 |
| RRT 1.04 | ND | ND | 0.07 | 0.07 | 0.07 | 0.07 |
| RRT 1.05 | 0.30 | 0.26 | 0.21 | 0.21 | 0.20 | 0.19 |
| RRT 1.055 | 0.16 | 0.15 | 0.17 | 0.15 | 0.15 | 0.16 |
| RRT 1.06 | <LOQ | <LOQ | ND | <LOQ | <LOQ | <LOQ |
| RRT 1.07 | 0.08 | 0.05 | 0.12 | 0.06 | 0.07 | <LOQ |
| RRT 1.09 | <LOQ | <LOQ | 0.05 | <LOQ | <LOQ | <LOQ |
| RRT 1.10 | <LOQ | <LOQ | <LOQ | <LOQ | <LOQ | <LOQ |
| RRT 1.11 | <LOQ | <LOQ | <LOQ | <LOQ | <LOQ | ND |
| RRT 1.12 | <LOQ | <LOQ | ND | ND | ND | ND |
| RRT 1.14 | <LOQ | <LOQ | <LOQ | <LOQ | <LOQ | <LOQ |
| RRT 1.16 | <LOQ | <LOQ | <LOQ | <LOQ | <LOQ | <LOQ |
| RRT 1.17 | <LOQ | <LOQ | ND | ND | ND | ND |
| RRT 1.19 | <LOQ | <LOQ | <LOQ | <LOQ | <LOQ | <LOQ |
| RRT 1.21 | 0.07 | 0.06 | 0.06 | 0.06 | 0.06 | 0.05 |
| Total¹ | 1.32 | 1.16 | 1.41 | 1.15 | 1.23 | 1.34 |

¹Sum of related substances >0.05%
ND = Not detected
LOQ = limit of quantification

TABLE 18
Purity/related substances (area %), LYT-02 40° C./75% RH

| | Time-point and amount (area %) | | | | | |
|---|---|---|---|---|---|---|
| | Pre- | | T = 2 | T = 5 | T = 7 | T = 14 |
| RRT | filtration | T = 0 | days | days | days | days |
| RRT 0.75 | 0.05 | <LOQ | <LOQ | <LOQ | <LOQ | <LOQ |
| RRT 0.88 | <LOQ | <LOQ | <LOQ | 0.05 | <LOQ | <LOQ |
| RRT 0.89 | <LOQ | <LOQ | <LOQ | <LOQ | <LOQ | 0.05 |
| RRT 0.90 | <LOQ | <LOQ | 0.05 | <LOQ | <LOQ | <LOQ |
| RRT 0.91 | 0.07 | 0.06 | <LOQ | <LOQ | <LOQ | 0.06 |
| RRT 0.94 | <LOQ | <LOQ | <LOQ | <LOQ | <LOQ | <LOQ |
| RRT 0.95 | <LOQ | <LOQ | 0.08 | 0.05 | <LOQ | <LOQ |
| RRT 0.955 | <LOQ | <LOQ | ND | <LOQ | <LOQ | 0.09 |
| RRT 0.96 | 0.08 | 0.09 | 0.11 | 0.09 | 0.07 | 0.10 |
| RRT 0.98 | 0.10 | 0.09 | 0.12 | 0.11 | 0.10 | 0.10 |
| LTX-315 | 98.40 | 98.53 | 98.19 | 98.22 | 98.27 | 97.99 |
| RRT 1.02 | 0.41 | 0.39 | 0.40 | 0.41 | 0.41 | 0.43 |
| RRT 1.04 | ND | ND | 0.10 | 0.12 | 0.11 | 0.22 |
| RRT 1.05 | 0.30 | 0.26 | 0.23 | 0.21 | 0.23 | 0.21 |
| RRT 1.055 | 0.16 | 0.15 | 0.22 | 0.18 | 0.20 | 0.19 |
| RRT 1.06 | <LOQ | <LOQ | ND | <LOQ | <LOQ | ND |
| RRT 1.07 | 0.08 | 0.05 | 0.13 | 0.07 | 0.06 | <LOQ |
| RRT 1.09 | <LOQ | <LOQ | 0.09 | 0.10 | 0.13 | 0.24 |
| RRT 1.10 | <LOQ | <LOQ | <LOQ | <LOQ | <LOQ | <LOQ |
| RRT 1.11 | <LOQ | <LOQ | <LOQ | ND | ND | <LOQ |
| RRT 1.12 | <LOQ | <LOQ | ND | ND | <LOQ | ND |
| RRT 1.14 | <LOQ | <LOQ | <LOQ | <LOQ | <LOQ | <LOQ |
| RRT 1.16 | <LOQ | <LOQ | <LOQ | <LOQ | <LOQ | <LOQ |
| RRT 1.17 | <LOQ | <LOQ | ND | ND | <LOQ | <LOQ |
| RRT 1.19 | <LOQ | <LOQ | <LOQ | <LOQ | <LOQ | <LOQ |
| RRT 1.21 | 0.07 | 0.06 | 0.06 | 0.07 | 0.07 | 0.06 |
| Total¹ | 1.32 | 1.16 | 1.60 | 1.45 | 1.38 | 1.53 |

¹Sum of related substances >0.05%
ND = Not detected
LOQ = limit of quantification

TABLE 19
Purity/related substances (area %), LYT-03 ambient

| | Time-point and amount (area %) | | | | | |
|---|---|---|---|---|---|---|
| | Pre- | | T = 2 | T = 5 | T = 7 | T = 14 |
| RRT | filtration | T = 0 | days | days | days | days |
| RRT 0.75 | <LOQ | <LOQ | <LOQ | <LOQ | <LOQ | <LOQ |
| RRT 0.88 | <LOQ | <LOQ | <LOQ | <LOQ | <LOQ | 0.06 |
| RRT 0.89 | <LOQ | <LOQ | <LOQ | <LOQ | <LOQ | 0.07 |
| RRT 0.90 | <LOQ | <LOQ | 0.06 | <LOQ | <LOQ | 0.05 |
| RRT 0.91 | <LOQ | 0.05 | <LOQ | <LOQ | 0.06 | 0.08 |
| RRT 0.94 | <LOQ | <LOQ | <LOQ | <LOQ | <LOQ | <LOQ |
| RRT 0.95 | <LOQ | <LOQ | 0.06 | 0.05 | <LOQ | <LOQ |
| RRT 0.955 | <LOQ | <LOQ | ND | <LOQ | <LOQ | 0.07 |
| RRT 0.96 | 0.07 | 0.08 | 0.11 | 0.09 | 0.08 | 0.09 |
| RRT 0.98 | 0.08 | 0.10 | 0.12 | 0.11 | 0.10 | 0.09 |
| LTX-315 | 98.54 | 98.39 | 98.19 | 98.45 | 98.57 | 98.37 |
| RRT 1.02 | 0.39 | 0.40 | 0.38 | 0.41 | 0.40 | 0.43 |
| RRT 1.04 | ND | ND | 0.07 | 0.14 | 0.07 | 0.07 |
| RRT 1.05 | 0.29 | 0.29 | 0.23 | 0.13 | 0.21 | 0.19 |
| RRT 1.055 | 0.16 | 0.16 | 0.18 | 0.16 | 0.18 | 0.15 |
| RRT 1.06 | <LOQ | <LOQ | <LOQ | <LOQ | ND | <LOQ |
| RRT 1.07 | 0.06 | 0.08 | 0.12 | 0.07 | 0.05 | 0.05 |
| RRT 1.09 | <LOQ | <LOQ | 0.05 | <LOQ | <LOQ | ND |
| RRT 1.10 | <LOQ | <LOQ | 0.05 | <LOQ | <LOQ | <LOQ |
| RRT 1.11 | <LOQ | <LOQ | <LOQ | <LOQ | <LOQ | <LOQ |
| RRT 1.12 | <LOQ | <LOQ | ND | ND | <LOQ | ND |
| RRT 1.14 | <LOQ | <LOQ | <LOQ | <LOQ | <LOQ | <LOQ |
| RRT 1.16 | <LOQ | <LOQ | <LOQ | <LOQ | <LOQ | <LOQ |
| RRT 1.17 | <LOQ | <LOQ | ND | ND | ND | ND |
| RRT 1.19 | <LOQ | <LOQ | <LOQ | <LOQ | <LOQ | <LOQ |
| RRT 1.21 | 0.06 | 0.09 | 0.07 | 0.06 | 0.06 | 0.05 |
| Total¹ | 1.10 | 1.26 | 1.50 | 1.22 | 1.21 | 1.44 |

¹Sum of related substances >0.05%
ND = Not detected
LOQ = limit of quantification

TABLE 20
Purity/related substances (area %), LYT-03 40° C./75% RH

| | Time-point and amount (area %) | | | | | |
|---|---|---|---|---|---|---|
| | Pre- | | T = 2 | T = 5 | T = 7 | T = 14 |
| RRT | filtration | T = 0 | days | days | days | days |
| RRT 0.75 | <LOQ | <LOQ | <LOQ | <LOQ | <LOQ | <LOQ |
| RRT 0.88 | <LOQ | <LOQ | <LOQ | 0.05 | <LOQ | <LOQ |
| RRT 0.89 | <LOQ | <LOQ | <LOQ | <LOQ | <LOQ | <LOQ |
| RRT 0.90 | <LOQ | <LOQ | <LOQ | 0.05 | <LOQ | <LOQ |
| RRT 0.91 | <LOQ | 0.05 | <LOQ | <LOQ | 0.06 | 0.07 |
| RRT 0.94 | <LOQ | <LOQ | <LOQ | <LOQ | <LOQ | <LOQ |
| RRT 0.95 | <LOQ | <LOQ | 0.12 | 0.09 | 0.10 | <LOQ |
| RRT 0.955 | <LOQ | <LOQ | ND | <LOQ | <LOQ | 0.14 |
| RRT 0.96 | 0.07 | 0.08 | 0.10 | 0.11 | 0.09 | 0.10 |
| RRT 0.98 | 0.08 | 0.10 | 0.11 | 0.13 | 0.12 | 0.07 |
| LTX-315 | 98.54 | 98.39 | 98.14 | 98.06 | 97.96 | 97.90 |
| RRT 1.02 | 0.39 | 0.40 | 0.42 | 0.43 | 0.46 | 0.48 |
| RRT 1.04 | ND | ND | 0.09 | 0.11 | 0.15 | 0.22 |
| RRT 1.05 | 0.29 | 0.29 | 0.22 | 0.22 | 0.20 | 0.16 |
| RRT 1.055 | 0.16 | 0.16 | 0.24 | 0.24 | 0.31 | 0.41 |
| RRT 1.06 | <LOQ | <LOQ | ND | <LOQ | <LOQ | ND |
| RRT 1.07 | 0.06 | 0.08 | 0.14 | 0.06 | 0.07 | <LOQ |
| RRT 1.09 | <LOQ | <LOQ | 0.08 | 0.07 | 0.10 | 0.15 |
| RRT 1.10 | <LOQ | <LOQ | <LOQ | <LOQ | <LOQ | <LOQ |
| RRT 1.11 | <LOQ | <LOQ | <LOQ | <LOQ | <LOQ | ND |
| RRT 1.12 | <LOQ | <LOQ | ND | ND | <LOQ | ND |
| RRT 1.14 | <LOQ | <LOQ | <LOQ | <LOQ | <LOQ | <LOQ |
| RRT 1.16 | <LOQ | <LOQ | <LOQ | <LOQ | <LOQ | <LOQ |
| RRT 1.17 | <LOQ | <LOQ | ND | ND | <LOQ | <LOQ |
| RRT 1.19 | <LOQ | <LOQ | <LOQ | <LOQ | <LOQ | <LOQ |
| RRT 1.21 | 0.06 | 0.09 | 0.06 | 0.06 | 0.07 | 0.05 |
| Total¹ | 1.10 | 1.26 | 1.58 | 1.61 | 1.71 | 1.87 |

¹Sum of related substances >0.05%
ND = Not detected
LOQ = limit of quantification

TABLE 21
Purity/related substances (area %), L YT-04 ambient

| | Time-point and amount (area %) | | | | | |
|---|---|---|---|---|---|---|
| | Pre- | | T = 2 | T = 5 | T = 7 | T = 14 |
| RRT | filtration | T = 0 | days | days | days | days |
| RRT 0.75 | <LOQ | <LOQ | <LOQ | <LOQ | <LOQ | <LOQ |
| RRT 0.86 | <LOQ | <LOQ | <LOQ | <LOQ | <LOQ | 0.05 |
| RRT 0.88 | <LOQ | <LOQ | <LOQ | <LOQ | <LOQ | <LOQ |
| RRT 0.89 | <LOQ | <LOQ | <LOQ | <LOQ | <LOQ | 0.07 |
| RRT 0.90 | <LOQ | <LOQ | 0.06 | <LOQ | <LOQ | <LOQ |
| RRT 0.91 | 0.05 | 0.05 | <LOQ | <LOQ | <LOQ | 0.10 |
| RRT 0.94 | <LOQ | <LOQ | <LOQ | <LOQ | <LOQ | 0.05 |
| RRT 0.95 | <LOQ | <LOQ | 0.09 | 0.08 | 0.08 | 0.05 |
| RRT 0.955 | <LOQ | <LOQ | ND | <LOQ | <LOQ | 0.08 |
| RRT 0.96 | 0.08 | 0.09 | 0.11 | 0.09 | 0.10 | 0.11 |
| RRT 0.98 | 0.08 | 0.11 | 0.12 | 0.12 | 0.10 | 0.13 |
| LTX-315 | 98.50 | 98.43 | 98.25 | 98.46 | 98.45 | 98.24 |
| RRT 1.02 | 0.40 | 0.40 | 0.40 | 0.43 | 0.43 | 0.43 |
| RRT L04 | ND | ND | 0.07 | 0.07 | 0.06 | 0.08 |
| RRT 1.05 | 0.28 | 0.28 | 0.22 | 0.20 | 0.21 | 0.19 |
| RRT 1.055 | 0.15 | 0.15 | 0.20 | 0.16 | 0.18 | 0.14 |
| RRT 1.06 | <LOQ | <LOQ | ND | <LOQ | ND | <LOQ |
| RRT 1.07 | 0.08 | 0.07 | 0.12 | 0.06 | 0.06 | 0.06 |
| RRT 1.09 | <LOQ | <LOQ | 0.06 | <LOQ | <LOQ | <LOQ |
| RRT 1.10 | <LOQ | <LOQ | <LOQ | <LOQ | <LOQ | <LOQ |
| RRT 1.11 | <LOQ | <LOQ | <LOQ | <LOQ | <LOQ | ND |
| RRT 1.12 | <LOQ | <LOQ | ND | ND | <LOQ | ND |
| RRT 1.13 | <LOQ | ND | <LOQ | <LOQ | <LOO | <LOQ |
| RRT 1.14 | <LOQ | <LOQ | <LOQ | <LOQ | <LOQ | <LOQ |
| RRT 1.16 | <LOQ | <LOQ | <LOQ | <LOQ | <LOQ | <LOQ |
| RRT 1.17 | <LOQ | <LOQ | ND | ND | <LOQ | ND |
| RRT 1.19 | <LOQ | 0.08 | <LOQ | <LOQ | <LOQ | <LOQ |
| RRT 1.21 | 0.06 | 0.08 | 0.06 | 0.06 | 0.06 | 0.05 |
| Total¹ | 1.16 | 1.22 | 1.50 | 1.25 | 1.22 | 1.58 |

¹Sum of related substances >0.05%
ND = Not detected
LOQ = limit of quantification

TABLE 22
Purity/related substances (area %), LYT-04 40° C./75% RH

| | Time-point and amount (area %) | | | | | |
|---|---|---|---|---|---|---|
| | Pre- | | T = 2 | T = 5 | T = 7 | T = 14 |
| RRT | filtration | T = 0 | days | days | days | days |
| RRT 0.75 | <LOQ | <LOQ | <LOQ | <LOQ | <LOQ | <LOQ |
| RRT 0.87 | <LOQ | <LOQ | <LOQ | <LOQ | 0.06 | <LOQ |
| RRT 0.88 | <LOQ | <LOQ | <LOQ | <LOQ | <LOQ | 0.11 |
| RRT 0.89 | <LOQ | <LOQ | <LOQ | <LOQ | <LOQ | <LOQ |
| RRT 0.90 | <LOQ | <LOQ | <LOQ | 0.06 | 0.10 | <LOQ |
| RRT 0.91 | 0.05 | 0.05 | <LOQ | <LOQ | 0.05 | 0.18 |
| RRT 0.94 | <LOQ | <LOQ | <LOQ | <LOQ | <LOQ | <LOQ |
| RRT 0.95 | <LOQ | <LOQ | 0.19 | 0.34 | 0.45 | <LOQ |
| RRT 0.955 | <LOQ | <LOQ | ND | <LOQ | <LOQ | 0.82 |
| RRT 0.96 | 0.08 | 0.09 | 0.11 | 0.10 | 0.10 | 0.15 |
| RRT 0.98 | 0.08 | 0.11 | 0.10 | 0.10 | 0.09 | 0.12 |
| LTX-315 | 98.50 | 98.43 | 97.95 | 97.63 | 97.34 | 96.40 |
| RRT 1.02 | 0.40 | 0.40 | 0.46 | 0.59 | 0.66 | 0.90 |
| RRT 1.04 | ND | ND | 0.09 | 0.10 | 0.14 | 0.20 |
| RRT 1.05 | 0.28 | 0.28 | 0.22 | 0.19 | 0.15 | 0.13 |
| RRT 1.055 | 0.15 | 0.15 | 0.27 | 0.36 | 0.43 | 0.64 |
| RRT 1.06 | <LOQ | <LOQ | ND | <LOQ | <LOQ | ND |
| RRT 1.07 | 0.08 | 0.07 | 0.14 | 0.07 | <LOQ | <LOQ |
| RRT 1.09 | <LOQ | <LOQ | 0.05 | <LOQ | <LOQ | 0.08 |
| RRT 1.10 | <LOQ | <LOQ | 0.06 | <LOQ | <LOQ | <LOQ |
| RRT 1.11 | <LOQ | <LOQ | <LOQ | <LOQ | <LOQ | ND |
| RRT 1.12 | <LOQ | <LOQ | ND | ND | ND | ND |
| RRT 1.13 | <LOQ | ND | <LOQ | <LOQ | <LOQ | <LOQ |
| RRT 1.14 | <LOQ | <LOQ | <LOQ | <LOQ | <LOQ | <LOQ |
| RRT 1.16 | <LOQ | <LOQ | <LOQ | <LOQ | <LOQ | <LOQ |
| RRT 1.17 | <LOQ | <LOQ | ND | ND | <LOQ | <LOQ |
| RRT 1.19 | <LOQ | 0.08 | <LOQ | <LOQ | <LOQ | <LOQ |
| RRT 1.21 | 0.06 | 0.08 | 0.06 | 0.06 | 0.07 | 0.05 |
| Total¹ | 1.16 | 1.22 | 1.75 | 1.93 | 2.31 | 3.37 |

¹Sum of related substances >0.05%
ND = Not detected
LOQ = limit of quantification

### Conclusions

The results in Example 2 show that LTX-315 is stable in an aqueous solution in a sealed container at pH between 5 and 7. At pH 8, an increase in related substances, which is also reflected by a decrease in concentration and purity, is seen.

### Example 3: Aqueous Formulations for Use in the Medical Product of the Invention

The aqueous formulations in this Example 3 can be manufactured using conventional preparation methods, including the following:
1. Weigh the buffer ingredients, prepare the amount of aqueous buffer needed and set aside.
2. Add approximately half of the amount of buffer to a vessel equipped with stirring means and optionally heating means, e.g. a flask and a magnetic stirrer, and stir.
3. Add the excipients to the buffer while stirring.
4. Finally, add the rest of the buffer, stir vigorously and continue stirring until complete dissolution of the excipients.

The peptide can be added in step 2 or step 4. In step 2, the buffer may be heated to about 70° C. to promote dissolution of certain excipients. If the buffer is heated, the remainder of the buffer in step 4 is preferably room temperature and the peptide is preferably added in step 4. Sterile formulations can be prepared by filtering the formulation through a 0.22 μm sterile filter in a container and sealing the container.

3a. 12 mg/ml LTX-315 Aqueous Formulation Comprising the Viscosity Enhancer HPMC (Low Molecular Weight).

HPMC was obtained from Safic Alcan, Paris La Defense Cedex, France.

TABLE 23

| Ingredients | Weight (g) | % w/w |
|---|---|---|
| LTX-315 acetate | 1.2 | 1.0 |
| HPMC low molecular weight | 0.5 | 0.5 |
| Acetic acid/acetate buffer 0.2M, pH 5.8 | Ad 100 | 98 |

The pH of the resulting aqueous formulation was 5.8, the viscosity 45 cP and the osmolarity 380 mmol/L.

3b. 6 mg/ml LTX-315 Aqueous Formulation Comprising the Viscosity Enhancer HPMC (High Molecular Weight).

HPMC was obtained from Safic Alcan, Paris La Defense Cedex, France.

TABLE 24

| Ingredients | Weight (g) | % w/w |
|---|---|---|
| LTX-315 acetate | 0.6 | 0.6 |
| HPMC high molecular weight | 0.5 | 0.5 |
| Acetic acid/acetate buffer 0.2M, pH 5.8 | Ad 100 | 99 |

The pH of the resulting aqueous formulation was 5.8, the viscosity 150 cP and the osmolarity 380 mmol/L.

3c. 10 mg/ml LTX-315 Aqueous Formulation Comprising the Viscosity Enhancer Chitosan (Low Molecular Weight).

Chitosan was obtained from Chitinor AS, Tromsø, Norway.

TABLE 25

| Ingredients | Weight (g) | % w/w |
|---|---|---|
| LTX-315 acetate | 1.0 | 1.0 |
| Chitosan low molecular weight | 1.0 | 1.0 |
| Acetic acid/acetate buffer 0.2M, pH 5.2 | Ad 100 | 98 |

The pH of the resulting aqueous formulation was 5.6, the viscosity 60 cP and the osmolarity 350 mmol/L.

3d. 16 mg/ml LTX-315 Aqueous Formulation Comprising the Viscosity Enhancer Chitosan (Medium Molecular Weight).

Chitosan was obtained from Chitinor AS, Tromsø, Norway.

TABLE 26

| Ingredients | Weight (g) | % w/w |
|---|---|---|
| LTX-315 acetate | 1.6 | 1.6 |
| Chitosan medium molecular weight | 1.0 | 1.0 |
| Acetic acid/acetate buffer 0.2M, pH 5.2 | Ad 100 | 97 |

The pH of the resulting aqueous formulation was 5.6, the viscosity 120 cP and the osmolarity 350 mmol/L.

3e. 16 mg/ml LTX-302 Aqueous Formulation Comprising the Viscosity Enhancer Chitosan (Medium Molecular Weight) and the Preservative Agent Benzyl Alcohol

TABLE 27

| Ingredients | Weight (g) | % w/w |
|---|---|---|
| LTX-302 acetate | 1.6 | 1.6 |
| Chitosan medium molecular weight | 0.5 | 0.5 |
| Benzyl alcohol | 1.0 | 1.0 |
| Phosphate buffer 0.2M, pH 6.0 | Ad 100 | 97 |

3f. 8 mg/ml LTX-302 Aqueous Formulation Comprising the Viscosity Enhancer Chitosan (Low Molecular Weight) and the Chelating Agent Disodium EDTA

TABLE 28

| Ingredients | Weight (g) | % w/w |
|---|---|---|
| LTX-302 acetate | 0.8 | 0.8 |
| Chitosan low molecular weight | 0.5 | 0.5 |
| Disodium EDTA | 0.1 | 0.1 |
| Phosphate buffer 0.2M, pH 6.0 | Ad 100 | 99 |

3g. 10 mg/ml LTX-320 Aqueous Formulation Comprising the Antioxidant Sodium Bisulfite

TABLE 29

| Ingredients | Weight (g) | % w/w |
|---|---|---|
| LTX-320 trifluoroacetate | 1.0 | 1.0 |
| Sodium bisulfite | 0.1 | 0.1 |
| Citric acid buffer 0.2M, pH 6.0 | Ad 100 | 99 |

3h. 10 mg/ml LTX-313 Aqueous Formulation Comprising the Stabilizer PEG 200

TABLE 30

| Ingredients | Weight (g) | % w/w |
|---|---|---|
| LTX-313 acetate | 1.0 | 0.9 |
| PEG 200 | 5.0 | 4.7 |
| Citric acid buffer 0.2M, pH 6.0 | Ad 100 | 94 |

### Example 4: Stability Investigations

A stability study of LTX-315 in different formulations, stored at 23±2° C. and at 2-8° C., has been conducted by Vitas Analytical Services, Norway. The sterile filtered formulations prepared were stored in 6R vials, Ph. Eur. Type I (Schott AG), stoppered with lyophilization rubber stopper V9172 FM257/2 ISAF1 Ph. Eur. Type I (Datwyler Pharma Packaging) and capped with a flip-off aluminium cap (West Pharmaceutical Services). The formulations were stored at 2-8° C. and 23±2° C. The parameters assay, purity, related impurities, visual appearance, and pH were tested for up to 4 months of storage.

The formulations are summarised below, Formulations 3-6 contain 10 mg/ml LTX-315 (net peptide):

| | | |
|---|---|---|
| Formulation 1 | Acetate buffer 0.1M, pH 5.2 | |
| Formulation 2 | Phosphate buffer 0.1M, pH 6 | |
| Formulation 3 | Acetate buffer 0.1M, pH 5.2 | Disodium EDTA 0.1% |
| | | Benzyl alcohol 1% |
| Formulation 4 | Acetate buffer 0.1M, pH 5.2 | PEG-400 2% |
| Formulation 5 | Acetate buffer 0.1M, pH 5.2 | Chitosan 0.4% |
| Formulation 6 | Acetate buffer 0.1M, pH 5.2 | HPMC 0.2% |

### Materials

### Preparation of the Buffers:

Acetate buffer 0.1 M pH 5.2:

| Composition | | |
|---|---|---|
| Acetic acid glacial | 1.3 | g |
| Sodium acetate 3-hydrate | 11.77 | g |
| NaOH/HCl | q.s. | |
| WFI | 1000 | ml |

q.s.: quantum satis

Phosphate buffer 0.1 M pH 6.0:

| Composition | |
|---|---|
| Disodium phosphate dihydrate | 8.9 g |
| Potassium dihydrogen phosphate | 6.8 g |
| NaOH/HCl | q.s. |
| WFI | Ad 1000 ml |

The buffers were filtered through a 0.22 μm sterile filter into flasks which have been rinsed with WFI which had been filtered through a 0.22 μm sterile filter or into flasks which had been autoclaved.

### Preparation of Formulations:

LTX-315 was provided in sealed vials containing 20 mg sterile, lyophilized peptide in the form of its acetate salt.

### Formulation 1

LTX-315 10 mg/ml

| Composition | | | % w/w |
|---|---|---|---|
| LTX-315 | 20 | mg | 1.0 |
| Acetate buffer 0.1M pH 5.2 | 2.0 | ml | 99 |

### Formulation 2

LTX-315 10 mg/ml

| Composition | | | % w/w |
|---|---|---|---|
| LTX-315 | 20 | mg | 1.0 |
| Phosphate buffer 0.1M pH 6.0 | 2.0 | ml | 99 |

For Formulation 1 and 2, 2 ml of the respective sterile buffer were injected into a vial comprising LTX-315, and LTX-315 was dissolved in the buffer. 2 vials were prepared for use in the stability investigation, where one vial at was stored at 2-8° C. and one at 23±2° C.

### Formulation 3

LTX-315 10 mg/ml

| Composition | | | % w/w |
|---|---|---|---|
| LTX-315 | 20 | mg | 1.0 |
| Disodium EDTA | 0.002 | g | 0.1 |
| Benzyl alcohol | 0.02 | g | 1.0 |

| | | |
|---|---|---|
| Acetate buffer 0.1M pH 5.2 | Ad 2 ml | 98 |

### Formulation 4

LTX-315 10 mg/ml

| Composition | | | % w/w |
|---|---|---|---|
| LTX-315 | 20 | mg | 1.0 |
| PEG-400 | 0.04 | g | 2.0 |

| | | |
|---|---|---|
| Acetate buffer 0.1M pH 5.2 | Ad 2 ml | 97 |

### Formulation 5

LTX-315 10 mg/ml

| Composition | | | % w/w |
|---|---|---|---|
| LTX-315 | 20 | mg | 1.0 |
| Chitosan | 0.008 | g | 0.4 |

| | | |
|---|---|---|
| Acetate buffer 0.1M pH 5.2 | Ad 2 ml | 98.6 |

Chitosan: low molecular weight, 330 kDa, DAA 86% (manufacturer Chitinor, Norway).

### Formulation 6

LTX-315 10 mg/ml

| Composition | | | % w/w |
|---|---|---|---|
| LTX-315 | 20 | mg | 1.0 |
| HPMC | 0.004 | g | 0.2 |

| | | |
|---|---|---|
| Acetate buffer 0.1M pH 5.2 | Ad 2 ml | 99 |

HPMC: about 86 kDA (manufacturer: Sigma Aldrich)

For Formulations 3-6, a preparation of the excipients in the acetate buffer was prepared as described in Example 3 and filtered through a 0.22 μm sterile filter. 2 ml of the respective sterile preparation were injected into a vial comprising LTX-315, and LTX-315 was dissolved in the preparation. 2 vials were prepared for use in the stability investigation, where one vial at was stored at 2-8° C. and one at 23±2° C.

### Assay, Purity, and Related Impurities

### Methods

The analytical method used in the stability study was an HPLC-UV method with external standard calibration and detection at 220 nm. The formulations were analysed using a Waters Acquity Cortecs C18+, 1.7 μm, 150×3.0 mm ID column at 15° C. and 0.612 mL/min with a mobile phase gradient of ACN/H₂O and a run time of 38 minutes. The injection volume was 2.0 μL and the injection temperature was 5° C.

The purity was calculated by subtracting the impurities measured (total organic impurities) from 100, and the related impurities were reported by comparing the diluent chromatogram with the sample chromatogram. Known peaks that were present in the diluent chromatogram with the same intensity for both solutions were disregarded. The method has been verified and is suitable for its intended purpose.

The pH was determined according to Ph. Eur. 2.2.3.

The appearance (clarity and coloration) was determined by visual inspection.

### Results and Conclusions

The pH and the appearance remained essentially unchanged for all formulations at each storage condition after 4 months.

No significant changes are observed in the assay after 4 months storage at each storage condition for any formulation at 2-8° C. or 23±2° C. storage condition, as presented in FIGS. 1 and 2. The small changes observed are due to method variability.

At the initial time point (TO), no impurities were observed for any of the formulations, thus the purity observed is 100% for all formulations. During storage for up to 4 months, there were no significant changes observed for any of the formulations at 2-8° C., as presented in FIG. 3.

For the formulations stored at 23±2° C., a decrease in purity to 98.4% was observed for Formulation 4 after 1 month (T1) and a further decrease in purity to 97.6% was observed after 4 months (T4), as presented in FIG. 4. The decrease in purity corresponds to the increase in total related impurities of 1.6% and 2.4% after 1 and 4 months, respectively.

A decrease in purity to 98.6% was observed for Formulation 3 after 4 months when stored at 23±2° C., which corresponds to the increase in total related impurities to 1.4%.

### Conclusion

Based on the above, LTX-315 is stable in different aqueous solutions at 2-8° C. in a sealed container as there are essentially no changes in any of the parameters tested. At 23±2° C., the stability results indicate some instabilities for Formulations 3 and 4, although these are not considered as a “significant change” according to ICH Q1A (Stability Testing of new Drug Substances and Products).

### EMBODIMENTS

1. A medical product in the form of a sealed container containing an aqueous formulation of a peptide, or a pharmaceutically acceptable salt thereof, which:
a) consists of 9 amino acids in a linear arrangement;
b) of those 9 amino acids, 5 are cationic and 4 are lipophilic; and
c) of these 4 lipophilic amino acids, 3 are tryptophan and 1 is non-genetically coded.
2. The medical product of embodiment 1, wherein the cationic amino acids in the peptide are lysine or arginine.
3. The medical product of any one of embodiments 1 to 2, wherein the non-genetically coded amino acid in the peptide is diphenylalanine or biphenylalanine.
4. The medical product of any one of embodiments 1 to 3, wherein the peptide has an amino acid sequence selected from SEQ ID NOs: 1, 2, 3, 4, 5, 6, 7, 8 and 9.
5. The medical product of any one of embodiments 1 to 4, wherein the peptide has an amino acid sequence selected from SEQ ID NOs: 1, 2, and 7.
6. The medical product of any one of embodiments 1 to 5, wherein the peptide has an amino acid sequence selected from SEQ ID NOs: 10 to 42.
7. The medical product of any one of embodiments 1 to 6, wherein the peptide is selected from the group consisting of LTX-302, LTX-313, LTX-315, LTX-320 and LTX-329.
8. The medical product of any one of embodiments 1 to 7, wherein the peptide is in the form of a pharmaceutically acceptable salt.
9. The medical product of any one of embodiments 1 to 8, wherein the peptide is in the form of an acetate salt.
10. The medical product of any one of embodiments 1 to 9, wherein the peptide is LTX-315 having the amino acid sequence of SEQ ID NO: 23.
11. The medical product of any one of embodiments 1 to 10, wherein the peptide is LTX-315 having the amino acid sequence of SEQ ID NO: 23 and is in salt form.
12. The medical product of any one of embodiments 1 to 11, wherein the peptide is LTX-315 having the amino acid sequence of SEQ ID NO: 23 and is in the form of its acetate salt.
13. The medical product of any one of embodiments 1 to 12, wherein the peptide is present in the aqueous formulation at a concentration of 1 to 30 mg/ml, preferably 4 to 20 mg/ml, or more preferably 6 to 16 mg/ml, such as 10 mg/ml.
14. The medical product of embodiment 13, wherein the amount of peptide is the net peptide amount.
15. The medical product of any one of embodiments 1 to 14, wherein the aqueous formulation comprises water for injection.
16. The medical product of any one of embodiments 1 to 15, wherein the aqueous formulation consists of the peptide and water for injection.
17. The medical product of any one of embodiments 1 to 15, wherein the aqueous formulation comprises an aqueous buffer, such as a buffer selected from the group consisting of acetic acid buffer, acetate buffer, aspartic acid buffer, sodium benzoate buffer, benzoic acid buffer, carbonic acid buffer, citric acid buffer, glucono-delta-lactone buffer, glycine buffer, glycine HCl buffer, histidine buffer, histidine HCl buffer, hydrobromic acid buffer, phosphate buffer (such as PBS), sodium succinate buffer, disodium succinate buffer, succinic acid buffer, sulfuric acid buffer, tartaric acid buffer and sodium tartrate buffer, preferably from the group consisting of acetic acid/acetate buffer, citric acid buffer and phosphate buffer.
18. The medical product of embodiment 17, wherein the aqueous buffer is a mixture of several different buffers.
19. The medical product of any one of embodiments 1 to 15 and 17 to 18, wherein the aqueous formulation consists of the peptide and an aqueous buffer.
20. The medical product of any one of embodiments 1 to 15 and 17 to 18, wherein the aqueous formulation comprises a salt or several salts.
21. The medical product of any one of embodiments 1 to 15, 17 to 18 and 20, wherein the aqueous formulation further comprises at least one preservative agent.
22. The medical product of embodiment 21, wherein the preservative agent is selected from the group consisting of benzalkonium chloride, benzyl alcohol, chlorobutanol, metacresol, methylparaben, phenol, potassium sorbate, propylparaben and thimerosal.
23. The medical product of any one of embodiments 1 to 15, 17 to 18 and 20 to 22, wherein the aqueous formulation further comprises at least one chelating agent.
24. The medical product of embodiment 23, wherein the chelating agent is selected from the group consisting of calcium disodium EDTA, calcium versetamide sodium, calteridol, disodium EDTA, DTPA and sodium EDTA.
25. The medical product of any one of embodiments 1 to 15, 17 to 18 and 20 to 24, wherein the aqueous formulation further comprises at least one antioxidant.
26. The medical product of embodiment 25, wherein the antioxidant is selected from the group consisting of ascorbic acid, ascorbyl palmitate, citric acid, erythorbic acid, methionine, monothioglycerol, potassium metabisulfite, sodium ascorbate, sodium bisulfite, sodium formaldehyde sulfoxylate, sodium thiosulfate and thiourea.
27. The medical product of any one of embodiments 1 to 15, 17 to 18 and 20 to 26, wherein the aqueous formulation further comprises at least one tonicity modifier.
28. The medical product of embodiment 27, wherein the tonicity modifier is selected from the group consisting of dextrose, glycerine, lactose, mannitol, potassium chloride, sodium chloride and sorbitol, perferably sodium chloride.
29. The medical product of any one of embodiments 1 to 15, 17 to 18 and 20 to 28, wherein the aqueous formulation further comprises at least one stabilizer.
30. The medical product of embodiment 29, wherein the stabilizer is selected from the group consisting of dimethyl sulfoxide, dimethylacetamide, ethanol, glycerol, mannitol, N-methyl-2-pyrrolidone, polyethylene glycol (PEG) 200, PEG 300, PEG 350, PEG 400, PEG 600, propylene glycol and sorbitol.
31. The medical product of any one of embodiments 1 to 15, 17 to 18 and 20 to 28, wherein the aqueous formulation further comprises at least one viscosity enhancer.
32. The medical product of embodiment 29, wherein the viscosity enhancer is selected from the group consisting of methylcellulose, hydroxypropyl cellulose, hydroxypropyl methylcellulose (HPMC), hydroxyethyl cellulose, carboxymethylcellulose calcium, carboxymethylcellulose sodium, chitosan, gelatine, hyaluronic acid, maltodextrin, pectin, poloxamer 407, PEG, polyethylene oxide, povidone (PVP), starches like corn starch and tapioca starch, tragacanth and β-cyclodextrin.
33. The medical product of embodiment 32, wherein the viscosity enhancer is chitosan or a chitosan derivative.
34. The medical product of any one embodiments 1 to 33, wherein the pH of the aqueous formulation is from 5.0 to 7.4, preferably from 5.0 to 7.0, and more preferably from 5.0 to 6.0, such as 6.5.
35. The medical product of any one embodiments 1 to 34, wherein the aqueous formulation has a viscosity of from 1, 1.5, 2, 2.5 or 3 cP to 300 cP, e.g. from 2.5 to 250 cP, such as 5 to 100 cP or 10 to 200 cP and 20 to 240 cP, preferably of from 40 to 180 cP, e.g. from 45 to 150 cP or from 30 to 100 cP and more preferably 1.5 to 10 cP, such as 2, 3, 4, 5, 6, 7, 8, 9 or 10 cP or of from 10 to 15 cP or 15 to 20 cP or 20 to 35 cP or 35 to 50 cP or 50 to 100 cP.
36. The medical product of any one of embodiments 1 to 35, wherein the total amount of excipients present in the aqueous formulation is no more than 20% w/w, preferably no more than 5% w/w, more preferably no more than 4% w/w, e.g. no more than 3% w/w or no more than 2% w/w.
37. The medical product of any one of embodiments 1 to 36, wherein the aqueous formulation comprises at least one organic excipient and the total amount of all such organic excipients is less than 10% w/w, less than 9% w/w, less than 8% w/w, less than 7% w/w, or less than 6% w/w, preferably less than 5% w/w, less than 4% w/w, less than 3% w/w, or less than 2% w/w or less than 1% w/w.
38. The medical product of any one of embodiments 1 to 37, wherein the aqueous formulation is a sterile formulation.
39. The medical product of any one of embodiments 1 to 38, wherein the aqueous formulation is a ready-to-inject formulation, preferably a ready-to-inject solution of the peptide.
40. The medical product of any one of embodiments 1 to 39, wherein the container is sealed with a manufactured seal.
41. The medical product of any one of embodiments 1 to 40, wherein the container is selected from the group consisting of pre-filled syringe, syringe cartridge, vial and ampoule.
42. The medical product of any one of embodiments 1 to 41, wherein the sealed container has a useable volume of 0.1 to 20 ml, such as of 0.2 to 10 ml, such as of 0.3 to 8 ml, preferably of 0.5 to 5 ml.
43. The medical product of any one of embodiments 1 to 42, wherein the sealed container contains a volume of the aqueous formulation of 0.1 to 20 ml, such as of 0.2 to 10 ml, preferably of 0.2 to 5.0 ml or 10 ml.
44. The medical product of any one of embodiments 1 to 43, wherein the sealed container contains a single dose.
45. The medical product of any one of embodiments 1 to 43, wherein the sealed container contains multiple doses.
46. A method of making the medical product according to any one of embodiments 1 to 45, wherein the aqueous formulation is introduced into the container and the filled container is mechanically sealed.
47. A method of treating a tumour in a subject, comprising obtaining the medical product according to any one of embodiments 1 to 45 and administering the aqueous formulation contained therein to said subject.
48. The method according to embodiment 47, wherein the aqueous formulation is administered into the tumour of the subject.
49. The method according to any one of embodiments 47 to 48, wherein the tumour is selected from the group consisting of carcinoma (e.g. hepatocellular carcinoma), adenocarcinoma, sarcoma, melanoma, lymphoma, leukemia, germ cell tumour, blastoma and glioblastoma and are related to cancers selected from the group consisting of neurological cancer, brain cancer, breast cancer, colon cancer, kidney cancer, liver cancer lung cancer, ovary cancer, pancreas cancer, prostate cancer head and neck cancer.
50. The method according to embodiment 49, wherein the tumour is selected from the group consisting of melanoma, carcinoma, sarcoma and lymphoma and related to cancers selected from skin cancer, breast cancer and head and neck cancer.

1. A medical product in the form of a sealed container containing an aqueous formulation of a peptide, or a pharmaceutically acceptable salt thereof, which:
a) consists of 9 amino acids in a linear arrangement;
b) of those 9 amino acids, 5 are cationic and 4 are lipophilic; and
c) of these 4 lipophilic amino acids, 3 are tryptophan and 1 is non-genetically coded.

2. The medical product of claim 1, wherein the cationic amino acids in the peptide are lysine or arginine and wherein the non-genetically coded amino acid in the peptide is diphenylalanine or biphenylalanine.

3. The medical product of any one of claims 1 to 2, wherein the peptide is selected from the group consisting of LTX-302, LTX-313, LTX-315, LTX-320 and LTX-329 and is preferably in the form of a pharmaceutically acceptable salt, preferably an acetate salt.

4. The medical product of any one of claims 1 to 3, wherein the peptide is LTX-315 having the amino acid sequence of SEQ ID NO: 23 and is in salt form, preferably an acetate salt.

5. The medical product of any one of claims 1 to 4, wherein the peptide is present in the aqueous formulation at a concentration of 1 to 30 mg/ml, preferably 4 to 20 mg/ml.

6. The medical product of any one of claims 1 to 5, wherein the aqueous formulation comprises water for injection or an aqueous buffer.

7. The medical product of any one of claims 1 to 6, wherein the aqueous formulation consists of the peptide and water, preferably water for injection, or consists of the peptide and an aqueous buffer.

8. The medical product of any one of claims 1 to 7, wherein the aqueous formulation further comprises at least one excipient selected from the group consisting of: a salt, a preservative agent, a tonicity modifier, a stabilizer, an antioxidant, a viscosity enhancer and a chelating agent.

9. The medical product of any one of claims 1 to 8, wherein the aqueous formulation further comprises at least one viscosity enhancer.

10. The medical product of claim 9, wherein the viscosity enhancer is chitosan or a chitosan derivative.

11. The medical product of any one of claims 1 to 10, wherein the aqueous formulation has a viscosity of from 1 cP to 300 cP.

12. The medical product of any one of claims 1 to 11, wherein the pH of the aqueous formulation is from 5.0 to 7.4.

13. The medical product of any of claims 1 to 12, wherein the total amount of excipients present in the aqueous formulation is no more than 20% w/w, preferably no more than 5% w/w.

14. The medical product of any of claims 1 to 6 and 8 to 13, wherein the aqueous formulation comprises at least one organic excipient and the total amount of all such organic excipients is less than 10% w/w.

15. The medical product of any one of claims 1 to 14, wherein the aqueous formulation is a sterile solution of the peptide, preferably a sterile, ready-to-inject solution of the peptide.

16. The medical product of any one of claims 1 to 15, wherein the container is sealed with a manufactured seal.

17. The medical product of any one of claims 1 to 16, wherein the container is selected from the group consisting of pre-filled syringe, syringe cartridge, vial and ampoule.

18. The medical product of any one of claims 1 to 17, wherein the sealed container contains a volume of the aqueous formulation of 0.1 to 20 ml, preferably 0.2 to 10 ml or 0.2 to 5 ml.

19. The medical product of any one of claims 1 to 18, wherein the sealed container contains a single dose.

20. The medical product of any one of claims 1 to 18, wherein the sealed container contains multiple doses.

21. A method of making the medical product according to any one of claims 1 to 20, wherein the aqueous formulation is introduced into the container and the filled container is mechanically sealed.

22. A method of treating a tumour in a subject, comprising obtaining the medical product according to any one of claims 1 to 20 and administering the aqueous formulation contained therein to said subject.

23. The method according to claim 22, wherein the aqueous formulation is administered into the tumour of the subject.

24. The method according to any one of claims 22 to 23, wherein the tumour is selected from the group consisting of carcinoma (e.g. hepatocellular carcinoma), adenocarcinoma, sarcoma, melanoma, lymphoma, leukemia, germ cell tumour, blastoma and glioblastoma and are related to cancers selected from the group consisting of neurological cancer, brain cancer, breast cancer, colon cancer, kidney cancer, liver cancer lung cancer, ovary cancer, pancreas cancer, prostate cancer head and neck cancer.

25. The method according to any one of claims 22 to 24, wherein the tumour is selected from the group consisting of melanoma, carcinoma, sarcoma and lymphoma and related to cancers selected from skin cancer, breast cancer and head and neck cancer.

26. The use of a peptide, or a pharmaceutically acceptable salt thereof, which:
a) consists of 9 amino acids in a linear arrangement;
b) of those 9 amino acids, 5 are cationic and 4 are lipophilic; and
c) of these 4 lipophilic amino acids, 3 are tryptophan and 1 is non-genetically coded
for the manufacture of a medical product in the form of a sealed container containing an aqueous formulation of said peptide or pharmaceutically acceptable salt thereof, for treating a tumour in a subject, wherein the aqueous formulation contained in the sealed container is administered to the subject.


SEQUENCE LISTING (42 sequences)

SEQ ID NO: 1 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  VARIANT 3..4: note = W or a non-genetically coded lipophilic amino acid
  VARIANT 7..8: note = W or a non-genetically coded lipophilic amino acid
  VARIANT 1..9: note = three X positions are W and one is a non-genetically coded lipophilic amino acid
KKXXKKXXK

SEQ ID NO: 2 - protein, 9 aa, synthetic construct
  VARIANT 2..3: note = or R or H or any non-genetically coded or modified amino acid carrying a positive charge at pH 7.0
  VARIANT 6..7: note = or R or H or any non-genetically coded or modified amino acid carrying a positive charge at pH 7.0
  VARIANT 9: note = or R or H or any non-genetically coded or modified amino acid carrying a positive charge at pH 7.0
  VARIANT 1: note = W or a non-genetically coded lipophilic amino acid
  VARIANT 4..5: note = W or a non-genetically coded lipophilic amino acid
  VARIANT 8: note = W or a non-genetically coded lipophilic amino acid
  VARIANT 1..9: note = three X positions are W and one is a non-genetically coded lipophilic amino acid
XKKXXKKXK

SEQ ID NO: 3 - protein, 9 aa, synthetic construct
  VARIANT 1: note = or R or H or any non-genetically coded or modified amino acid carrying a positive charge at pH 7.0
  VARIANT 4..5: note = or R or H or any non-genetically coded or modified amino acid carrying a positive charge at pH 7.0
  VARIANT 8..9: note = or R or H or any non-genetically coded or modified amino acid carrying a positive charge at pH 7.0
  VARIANT 2..3: note = W or a non-genetically coded lipophilic amino acid
  VARIANT 6..7: note = W or a non-genetically coded lipophilic amino acid
  VARIANT 1..9: note = three X positions are W and one is a non-genetically coded lipophilic amino acid
KXXKKXXKK

SEQ ID NO: 4 - protein, 9 aa, synthetic construct
  VARIANT 1..2: note = or R or H or any non-genetically coded or modified amino acid carrying a positive charge at pH 7.0
  VARIANT 5: note = or R or H or any non-genetically coded or modified amino acid carrying a positive charge at pH 7.0
  VARIANT 8..9: note = or R or H or any non-genetically coded or modified amino acid carrying a positive charge at pH 7.0
  VARIANT 3..4: note = W or a non-genetically coded lipophilic amino acid
  VARIANT 6..7: note = W or a non-genetically coded lipophilic amino acid
  VARIANT 1..9: note = three X positions are W and one is a non-genetically coded lipophilic amino acid
KKXXKXXKK

SEQ ID NO: 5 - protein, 9 aa, synthetic construct
  VARIANT 1: note = or R or H or any non-genetically coded or modified amino acid carrying a positive charge at pH 7.0
  VARIANT 3..4: note = or R or H or any non-genetically coded or modified amino acid carrying a positive charge at pH 7.0
  VARIANT 7..8: note = or R or H or any non-genetically coded or modified amino acid carrying a positive charge at pH 7.0
  VARIANT 2: note = W or a non-genetically coded lipophilic amino acid
  VARIANT 5..6: note = W or a non-genetically coded lipophilic amino acid
  VARIANT 9: note = W or a non-genetically coded lipophilic amino acid
  VARIANT 1..9: note = three X positions are W and one is a non-genetically coded lipophilic amino acid
KXKKXXKKX

SEQ ID NO: 6 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 3: note = a non-genetically coded lipophilic amino acid
KKXWKKWWK

SEQ ID NO: 7 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 8: note = a non-genetically coded lipophilic amino acid
KKWWKKWXK

SEQ ID NO: 8 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 4: note = a non-genetically coded lipophilic amino acid
KKWXKKWWK

SEQ ID NO: 9 - protein, 9 aa, synthetic construct
  VARIANT 2..3: note = or R or H or any non-genetically coded or modified amino acid carrying a positive charge at pH 7.0
  VARIANT 6..7: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 5: note = a non-genetically coded lipophilic amino acid
WKKWXKKWK

SEQ ID NO: 10 - protein, 9 aa, synthetic construct
  SITE 1: note = diphenylalanine
  MOD_RES 9: note = AMIDATION
XKKWWKKWK

SEQ ID NO: 11 - protein, 9 aa, synthetic construct
  SITE 5: note = diphenylalanine
  MOD_RES 9: note = AMIDATION
WKKWXKKWK

SEQ ID NO: 12 - protein, 9 aa, synthetic construct
  SITE 8: note = diphenylalanine
  MOD_RES 9: note = AMIDATION
WKKWWKKXK

SEQ ID NO: 13 - protein, 9 aa, synthetic construct
  SITE 1: note = biphenylalanine
  MOD_RES 9: note = AMIDATION
XKKWWKKWK

SEQ ID NO: 14 - protein, 9 aa, synthetic construct
  SITE 4: note = biphenylalanine
  MOD_RES 9: note = AMIDATION
WKKXWKKWK

SEQ ID NO: 15 - protein, 9 aa, synthetic construct
  SITE 5: note = D-diphenylalanine
  SITE 1: note = D-tryptophan
  SITE 2..3: note = D-lysine
  SITE 4: note = D-tryptophan
  SITE 6..7: note = D-lysine
  SITE 8: note = D-tryptophan
  SITE 9: note = D-lysine
  MOD_RES 9: note = AMIDATION
WKKWXKKWK

SEQ ID NO: 16 - protein, 9 aa, synthetic construct
  SITE 4: note = diphenylalanine
  MOD_RES 9: note = AMIDATION
KKWXKKWWK

SEQ ID NO: 17 - protein, 9 aa, synthetic construct
  SITE 4: note = diphenylalanine
  SITE 1..2: note = D-lysine
  SITE 5..6: note = D-lysine
  SITE 9: note = D-lysine
  MOD_RES 9: note = AMIDATION
KKWXKKWWK

SEQ ID NO: 18 - protein, 9 aa, synthetic construct
  SITE 4: note = diphenylalanine
  MOD_RES 9: note = AMIDATION
KKWXKKWXK

SEQ ID NO: 19 - protein, 9 aa, synthetic construct
  SITE 4: note = biphenylalanine
  MOD_RES 9: note = AMIDATION
KKWXKKWWK

SEQ ID NO: 20 - protein, 9 aa, synthetic construct
  SITE 2: note = biphenylalanine
  MOD_RES 9: note = AMIDATION
KXKKWWKKW

SEQ ID NO: 21 - protein, 9 aa, synthetic construct
  SITE 3: note = biphenylalanine
  MOD_RES 9: note = AMIDATION
KKXWKKWWK

SEQ ID NO: 22 - protein, 9 aa, synthetic construct
  SITE 7: note = diphenylalanine
  MOD_RES 9: note = AMIDATION
KKWWKKXWK

SEQ ID NO: 23 - protein, 9 aa, synthetic construct
  SITE 8: note = diphenylalanine
  MOD_RES 9: note = AMIDATION
KKWWKKWXK

SEQ ID NO: 24 - protein, 9 aa, synthetic construct
  SITE 3: note = diphenylalanine
  MOD_RES 9: note = AMIDATION
KWXKKWWKK

SEQ ID NO: 25 - protein, 9 aa, synthetic construct
  SITE 7: note = diphenylalanine
  MOD_RES 9: note = AMIDATION
KKWWKWXKK

SEQ ID NO: 26-27 - skipped (2 entries)

SEQ ID NO: 28 - protein, 9 aa, synthetic construct
  SITE 4: note = diphenylalanine
  MOD_RES 9: note = AMIDATION
RRWXRRWWR

SEQ ID NO: 29 - protein, 9 aa, synthetic construct
  SITE 6: note = diphenylalanine
  MOD_RES 9: note = AMIDATION
KWWKKXWKK

SEQ ID NO: 30 - protein, 9 aa, synthetic construct
  SITE 2: note = diphenylalanine
  MOD_RES 9: note = AMIDATION
KXKKWWKKW

SEQ ID NO: 31 - protein, 9 aa, synthetic construct
  SITE 3: note = diphenylalanine
  MOD_RES 9: note = AMIDATION
KKXWKKWWK

SEQ ID NO: 32 - protein, 9 aa, synthetic construct
  SITE 6: note = D-diphenylalanine
  SITE 1: note = D-lysine
  SITE 2..3: note = D-tryptophan
  SITE 4..5: note = D-lysine
  SITE 7: note = D-tryptophan
  SITE 8..9: note = D-lysine
  MOD_RES 9: note = AMIDATION
KWWKKXWKK

SEQ ID NO: 33 - protein, 9 aa, synthetic construct
  SITE 3: note = biphenylalanine
  MOD_RES 9: note = AMIDATION
RRXWRRWWR

SEQ ID NO: 34 - protein, 9 aa, synthetic construct
  SITE 3: note = diphenylalanine
  MOD_RES 9: note = AMIDATION
RRXWRRWWR

SEQ ID NO: 35 - protein, 9 aa, synthetic construct
  SITE 3: note = D-biphenylalanine
  SITE 1..2: note = D-lysine
  SITE 4: note = D-tryptophan
  SITE 5..6: note = D-lysine
  SITE 7..8: note = D-tryptophan
  SITE 9: note = D-lysine
  MOD_RES 9: note = AMIDATION
KKXWKKWWK

SEQ ID NO: 36 - protein, 9 aa, synthetic construct
  SITE 3: note = biphenylalanine
  SITE 1..2: note = D-lysine
  SITE 4: note = D-tryptophan
  SITE 5..6: note = D-lysine
  SITE 7..8: note = D-tryptophan
  SITE 9: note = D-lysine
  MOD_RES 9: note = AMIDATION
KKXWKKWWK

SEQ ID NO: 37 - protein, 9 aa, synthetic construct
  SITE 3: note = D-biphenylalanine
  MOD_RES 9: note = AMIDATION
KKXWKKWWK

SEQ ID NO: 38 - skipped

SEQ ID NO: 39 - protein, 9 aa, synthetic construct
  SITE 4: note = 1-naphthylalanine
  MOD_RES 9: note = AMIDATION
KKWXKKWWK

SEQ ID NO: 40 - protein, 9 aa, synthetic construct
  SITE 4: note = 2-naphthylalanine
  MOD_RES 9: note = AMIDATION
KKWXKKWWK

SEQ ID NO: 41 - protein, 9 aa, synthetic construct
  SITE 4: note = 2-amino-3-(anthracen-9-yl)propanoic acid
  MOD_RES 9: note = AMIDATION
KKWXKKWWK

SEQ ID NO: 42 - protein, 9 aa, synthetic construct
  SITE 4: note = 2-amino-3-[1,1':4',1"-terphenyl-4-yl]propionic acid
  MOD_RES 9: note = AMIDATION
KKWXKKWWK